SEQUENCE LISTING (1 sequence)

SEQ ID NO: 1 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

(19) United States
(12) Patent Application Publication
(10) Pub. No.: US 2017/0100374 A1
(43) Pub. Date: Apr. 13, 2017

(54) POTENTIATOR-CORRECTOR COMBINATIONS USEFUL IN THE TREATMENT OF CYSTIC FIBROSIS

(71) Applicants: AbbVie S.à.r.l., Luxembourg (LU); Galapagos NV, Mechelen (BE)

(72) Inventors: Katja E. Conrath, Mechelen (BE); Steven M. Rowe, Birmingham, AL (US)

(21) Appl. No.: 15/288,249

(22) Filed: Oct. 7, 2016

Related U.S. Application Data
(60) Provisional application No. 62/239,667, filed on Oct. 9, 2015.

(51) Int. Cl. A61K 31/4155; G01N 33/68; C12Q 1/68; A61K 45/06; A61K 31/36

(52) U.S. Cl. CPC A61K 31/4155; A61K 45/06; A61K 31/36; C12Q 1/6883; G01N 33/6893; G01N 33/6872; C12Q 2600/156; G01N 2333/705; G01N 2800/382

(57) ABSTRACT

A combination therapy including a modulator of the function (potentiator) of cystic fibrosis transmembrane conductance regulator (CFTR) protein, and one or two modulator(s) of the cellular processing and/or localization molecule (correctors) is provided in a method for treating cystic fibrosis in a subject having a mutation located between the amino acid residues 1164-1480 of full length wild-type CFTR.

### CROSS REFERENCE TO RELATED APPLICATIONS

This application claims priority to U.S. Provisional Application No. 62/239,667, filed Oct. 9, 2015, which is incorporated herein by reference for all purposes.

### FIELD OF THE INVENTION

The present invention relates generally to the field of pharmacotherapy of genetic diseases. More specifically, the present invention relates to a novel treatment of cystic fibrosis using a combination therapy.

### BACKGROUND OF THE INVENTION

Cystic fibrosis (CF) is the most common autosomal recessive disorder in the Caucasian population. Approximately 1 in 25 Caucasian persons are carriers of the disease. The responsible gene has been localized on the long arm of chromosome 7. The sequence encodes a membrane-associated protein that was called the “cystic fibrosis transmembrane regulator” (“CFTR”). The CFTR gene contains 27 exons and encodes a protein of 1480 amino acids (Gregory et al., 1990; Rich et al., 1990). CFTR is a glycoprotein and classified as an ABC (ATP-binding cassette) transporter. The protein consists of five domains. There are two nucleotide binding domains (NBD1 and NBD2), a regulatory domain (RD) and two membrane spanning domains (MSD1 and MSD2). The protein activity is regulated by cAMP-dependent Protein Kinase (PKA) which catalyze phosphorylation of regulatory domain (RD) and also by binding of two ATP molecules to NBD1 and NBD2 domains (Riordan et al., 2005).

CFTR protein production starts in the nucleus of the cell when CFTR gene sequence is transcribed into RNA; splicing then occurs to form messenger RNA (mRNA). mRNA is transported from the nucleus to the endoplasmic reticulum (ER), where mRNA is translated into a protein and the protein folding occurs. From the ER the protein is transported to the cell membrane (MacDonald et al., 2007). The normal process of transcription and translation results in a normal amount of CFTR protein at the cell membrane and in a normal chloride transport activity.

Mutations in CFTR result in defective chloride ion transport and defective electrolyte transport. Over 2000 mutations of the CFTR gene have been identified, and the mutations can be classified based on their effect on the CFTR production and activity: Class I: result in (almost) complete absence of CFTR protein synthesis; Class II: result in arrested maturation and intracellular localization defect of the CFTR protein; Class III: result in inhibition of regulation with defective activation of the chloride ion transport function; Class IV: result in reduced conductance of chloride ions; and Class V: result in reduced CFTR protein synthesis. The most common mutation of the CFTR gene is deletion of phenylalanine in position 508 of the polypeptide chain (mutation F508del-CFTR), which is a Class II mutation.

In order to restore the function of CFTR in cells, different types of modulators can be used. Briefly, the treatment of cystic fibrosis patients requires different modulators of the mutated CFTR protein, namely “correctors” and/or “potentiators”, depending on the mutations of the CFTR gene, which divide the patients into genetically distinct sub-groups. In addition to these direct modulators of CFTR complementary medicaments such as those with an antibacterial action or an anti-inflammatory action are commonly used to relieve the symptoms.

CFTR potentiators improve the function of CFTR channels that have gating (Class III) or conductance (Class IV) mutations (Rogan et al., 2011). There is additional evidence from in vitro studies that CFTR potentiators may also enhance the function of CFTR channels with Class II mutations (Van Goor et al., 2009). Nevertheless, a potentiator can only have an effect if the expressed CFTR channel is already located on the cell membrane. Thus, CFTR potentiators alone are not able to treat Class I or II mutations, which are characterized by an absence or lack or synthesized CFTR protein. An example of a potentiator is VX-770, which is successful only in patients suffering from cystic fibrosis with a class III/IV defect such as e.g. G551D-CFTR gene defect, who represent 1-5% of all the cystic fibrosis patients (Van Goor et al, 2009), but has no significant therapeutic efficacy in patients having F508del-CFTR class II mutation (Flume et al., 2012). That points to the need for customized treatments for sub-groups of patients suffering from cystic fibrosis, and such treatment depends on the nature of the mutation in the CFTR gene and the resulting defect in the CFTR protein.

Corrector compounds are being used to treat Class II mutations, such as F508del-CFTR. An example of such corrector is VX-809. The mutated protein F508del-CFTR, in addition to the Class II mutation effect (decreased maturation and intracellular localization defect of the CFTR protein) also has reduced chloride ion conductance. Tests of a combination of the VX-809 corrector with the VX-770 potentiator to modulate the function of the mutated protein F508del-CFTR have been already performed and show an improved result in patients carrying class II mutation effect (www.clinicaltrials.gov, study code NCT0122521 1).

Although potentiators and correctors have different effects on CFTR function, there is a potential correlation between CFTR activity detected in humans by changes in sweat chloride levels and in vitro in primary human bronchial epithelial cells (Rowe et al., 2013). This confirms that the performance of compounds in preclinical models is useful to identify prospective potentiators and correctors.

Another class of more rare mutations in CFTR gene, Class I mutations, include premature termination codons (“PTC”) or stop codons (also called “nonsense mutations”). Nonsense mutations are responsible for about 10% of cystic fibrosis cases worldwide. However, in Israel, nonsense mutations are the cause of cystic fibrosis in most patients (Kerem et al., 1997). A PTC is defined as a stop codon located in the coding sequence of a gene, upstream from the normal stop codon. A nonsense mutation is a single point alteration in DNA that results in the inappropriate presence of a UAA, UAG, or UGA stop codon in the protein-coding region of the corresponding mRNA transcript. Whereas the normal stop codon stops the gene translation and enables a full-length wild type protein synthesis, the PTC prevents the wild-type protein synthesis and leads to the partial or full suppression of transcription of the mutated gene. In turn, the partial or total lack of CFTR protein leads to the pathology. Not all type I mutations result in complete loss of the CFTR protein in cells. Rowe et al, 2007 described experimental evidence that a certain subset of Class I mutations occurring after position 1164 exhibited membrane localization and retained low but detectable chloride channel function after enhanced expression in the presence of a read-through agent.

Premature stop codon suppressors, also called “read-through agents” are of interest for their potential to be used in the treatment of cystic fibrosis arising from Class I mutations. Aminoglycoside antibiotics were the first drugs demonstrated to suppress PTCs in disease-causing mutations, allowing the translation of full length proteins (Hermann et al., 2007). Howard et al. (Howard et al., 1996) described PTC suppression by the synthetic aminoglycoside geneticin (G418) to restore protein function in HeLa cells expressing nonsense codons in 1996. Studies using another agent gentamicin in patients with CF showed small changes in Nasal Potential Difference (NPD) values (Wilschanski et al., 2003). However, the inconvenience of parenteral administration and the potential for serious side effects preclude long-term systemic use of gentamicin for suppression of Class I mutations (Wilschanski et al., 2012).

Ataluren (clinical study code PTC124) also has the ability to facilitate read through of PTCs without exhibiting toxic effects at normal therapeutic doses (Wilschanski et al., 2003; Kerem et al., 2008). Although ataluren seems to be specific for premature stop codons, serious adverse effects could occur if the drug allows read through of correct stop codons. Ataluren also has the potential to disturb nonsense-mediated mRNA decay, which protects against harmful byproducts of premature stop codons.

Therefore, there is a need of a method of treatment of class I mutations, specifically in patients carrying a premature termination codon (PTC) or a nonsense mutations that occur after position 1164 of the CFTR protein.

The present invention addresses the need for alternative treatments of such mutations by providing a novel combination of a potentiator with one or two correctors that are able to restore the CFTR function in patients having class I mutation located between positions 1164-1480 of the full coding sequence of the wild-type CFTR without requiring additional administration of a read-through corrector molecule.

### SUMMARY OF THE INVENTION

The present invention provides that a combination of a potentiator compound with one or more non read-through correctors restores the functional activity of CFTR protein having class I mutation located between positions 1164-1480 of the full coding sequence of the wild-type CFTR protein in the absence of a read-through agent.

In one aspect the present invention provides a method of treatment of cystic fibrosis in a subject comprising the steps of:
a. analyzing the sequence of cystic fibrosis transmembrane conductance regulator (CFTR) protein from the subject for the presence of a premature termination codon (PTC) or a nonsense mutation,
b. identifying a subject having amutation located between the amino acid residues 1164-1480 of SEQ ID NO: 1, and
c. administering a combination comprising:
   i. a modulator of the function (P potentiator) of cystic fibrosis transmembrane conductance regulator (CFTR) protein,
   ii. a modulator of the cellular processing and/or localization (C corrector), wherein said C corrector is not a read-through corrector,
      wherein said combination does not comprise a read-through agent, and wherein said combination produces an additional transepithelial conductance (ΔGt) of at least 1 mS/cm2 as measured using transepithelial clap circuit assay (TECC assay) in the W1282X Fisher rat thyroid (FRT) cells.

More specifically, said C corrector is either C1 or C2 corrector as disclosed herein.

The present invention also provides a method of treatment of cystic fibrosis in a subject comprising the steps of:
a. analyzing the sequence of cystic fibrosis transmembrane conductance regulator (CFTR) protein from the subject for the presence of a premature termination codon (PTC) or a nonsense mutation,
b. identifying a subject having a mutation located between the amino acid residues 1164-1480 of SEQ ID NO: 1, and
c. administering a combination comprising:
   i. a modulator of the function (P potentiator) of cystic fibrosis transmembrane conductance regulator (CFTR) protein,
   ii. a modulator of the cellular processing and/or localization (C corrector), wherein said C corrector is not a read-through corrector, and wherein said C corrector is not acting through the membrane spanning domain 1 (MSD1) domain of CFTR,
      wherein said combination does not comprise a read-through agent. More particular said C corrector is a C2 corrector.

The combinations may further comprise a second modulator of the cellular processing and/or localization (second C corrector), wherein said second C corrector is also not a read-through corrector. More particular said C corrector is C2 corrector and said second C corrector is C1 corrector. More specifically said correctors bind to different parts of CFTR protein.

The present invention further discloses kits and methods of enhancing the activity of mutant CFTR suing the combinations of the present invention.

### BRIEF DESCRIPTION OF THE DRAWINGS

FIG. 1 shows various domains of wild-type CFTR protein.

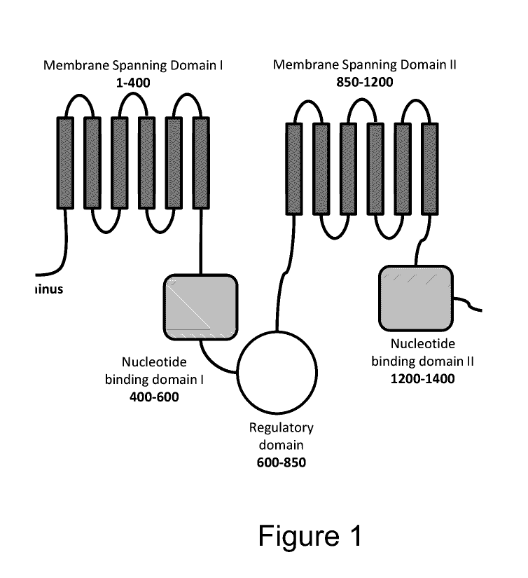

FIG. 2 shows the effect of correctors (C1 and C2) and a potentiator (GP-5) on the conductance in Fischer rat thyroid (FRT) cells. Effect of correctors and potentiator combinations in FRT CFTR PTC mutation W1282X: C1+C2 significantly more efficacious, alone and in combination with read-through agents. Cells were treated for 24 hour with either read-through agent and/or corrector C1/C2, the day after CFTR channel was activated using Fsk and potentiator GP-5 (* P<0.05, **P<0.01, *** P<0.001, **** P<0.0001)

FIG. 3 shows the effect of the correctors in a cell surface expression assay in CFBe41O-cell line. Dose response for C1 corrector, C2 corrector and a combination of C1 corrector with C2 corrector (fixed C1 corrector concentration).

FIG. 4 shows the effect of the potentiator in combination with C and C1 correctors with and without G418 (read-through agent) on the conductance in FRT cells containing W1282X CFTR mutation. In this graph, comparison was made between “acute” and “chronic” treatment with a potentiator (GP-5). In all conditions correctors C1/C2 or G418 were incubated on the cells for 24 hours, in the “acute” condition GP-5 was added after stimulation of CFTR channel with forskolin, in case of “chronic” treatment, GP-5 was also incubated for 24 hours together with corrector agents. Co-incubation of correctors and/or read-through agent together with GP-5 potentiator improves W1282X CFTR rescue (* P<0.05, **P<0.01, *** P<0.001, **** P<0.0001).

FIG. 5 shows the effect of correctors (C1 and C2) and/or read-through agent G418 and potentiator (GP-5) combination in primary human bronchial epithelial (HBE) cells with both delF508 and W1282X mutations. Channel activity is drastically improved when adding C1+C2 corrector mix combined with channel opening with GP-5 potentiator (* P<0.05, **P<0.01, *** P<0.001, **** P<0.0001).

FIG. 6 shows CFTR protein expression after 24 hour incubation of W1282X CFTR FRT cells with different combinations of potentiator, C1 corrector, C2 corrector and G418 agent. Higher bars indicate higher expression. Corrector combinations show increased CFTR protein levels.

### DETAILED DESCRIPTION OF THE INVENTION

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “a pharmaceutically acceptable carrier” means a single pharmaceutically acceptable carrier as well as one or more pharmaceutically acceptable carriers, and the like.

### DEFINITIONS

As used in the specification and the appended claims, unless specified to the contrary, the following terms have the meaning indicated:

The term “alkenyl” as used herein, means a straight or branched hydrocarbon chain containing from 2 to 10 carbons and containing at least one carbon-carbon double bond. The term “C₂-C₆ alkenyl” means an alkenyl group containing 2-6 carbon atoms. Non-limiting examples of C₂-C₆ alkenyl include buta-1,3-dienyl, ethenyl, 2-propenyl, 2-methyl-2-propenyl, 3-butenyl, 4-pentenyl, and 5-hexenyl.

The term “C1-C3 alkoxy” as used herein, means a C1-C3 alkyl group, as defined herein, appended to the parent molecular moiety through an oxygen atom. Examples of C1-C3 alkoxy include, but are not limited to, methoxy, ethoxy, propoxy, and 2-propoxy.

The term “alkyl” as used herein, means a saturated, straight or branched hydrocarbon chain radical. In some instances, the number of carbon atoms in an alkyl moiety is indicated by the prefix “Cx-Cy”, wherein x is the minimum and y is the maximum number of carbon atoms in the substituent. Thus, for example, “C1-C6 alkyl” means an alkyl substituent containing from 1 to 6 carbon atoms and “C1-C3 alkyl” means an alkyl substituent containing from 1 to 3 carbon atoms. Representative examples of C1-C6 alkyl include, but are not limited to, methyl, ethyl, n-propyl, iso-propyl, n-butyl, sec-butyl, iso-butyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl.

The term “alkylene” or “alkylenyl” means a divalent radical derived from a straight or branched, saturated hydrocarbon chain, for example, of 1 to 10 carbon atoms or of 1 to 6 carbon atoms (C1-C6 alkylenyl) or of 1 to 4 carbon atoms or of 1 to 3 carbon atoms (C1-C3 alkylenyl) or of 2 to 6 carbon atoms (C2-C6 alkylenyl). Examples of C1-C6 alkylenyl include, but are not limited to, —CH₂—, CH₂CH₂—, —C((CH₃)₂)—CH₂CH₂CH₂—, —C((CH₃)₂)CH₂CH₂, —CH₂CH₂CH₂CH₂—, and —CH₂CH(CH₃)CH₂—.

The term “C2-C6 alkynyl” as used herein, means a straight or branched chain hydrocarbon radical containing from 2 to 6 carbon atoms and containing at least one carbon-carbon triple bond. Representative examples of C2-C6 alkynyl include, but are not limited, to acetylenyl, 1-propynyl, 2-propynyl, 3-butynyl, 2-pentynyl, and 1-butynyl.

The term “cycloalkyl” as used herein, means a C3-C6 cycloalkyl as defined herein, wherein the C3-C6 cycloalkyl may further contain one or two alkylene bridges of 1, 2, 3, or 4 carbon atoms, and each links two non-adjacent carbon atoms of the ring. Examples of such bridged ring system include, but are not limited to, bicyclo[2.2.1]heptyl, bicyclo[2.1.1]hexyl, and bicyclo[3.1.1]heptyl. The cycloalkyl ring systems (including the exemplary rings) are optionally substituted unless otherwise indicated.

The term “C3-C6 cycloalkyl” as used herein, means cyclopropyl, cyclobutyl, cyclopentyl, and cyclohexyl, each of which is optionally substituted unless otherwise indicated.

The term “C4-C6 cycloalkenyl” as used herein, means cyclobutenyl, cyclopentenyl, and cyclohexenyl, each of which is optionally substituted unless otherwise indicated.

The term “halo” or “halogen” as used herein, means Cl, Br, I, and F.

The term “C1-C3 haloalkoxy” as used herein, means a C1-C3 haloalkyl group, as defined herein, appended to the parent molecular moiety through an oxygen atom. Examples of C1-C3 haloalkoxy include, but are not limited to, trifluoromethoxy, difluoromethoxy, and 2-fluoroethoxy.

The term “haloalkyl” as used herein, means an alkyl group, as defined herein, in which one, two, three, four, five or six hydrogen atoms are replaced by halogen. The term “C1-C6 haloalkyl” means a C1-C6 alkyl group, as defined herein, in which one, two, three, four, five, or six hydrogen atoms are replaced by halogen. The term “C1-C3 haloalkyl” means a C1-C3 alkyl group, as defined herein, in which one, two, three, four, or five hydrogen atoms are replaced by halogen. Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, 2-chloro-3-fluoropentyl, trifluorobutyl, and trifluoropropyl.

The term “heterocycle” or “heterocyclic” as used herein, means a radical of a monocyclic heterocycle, a bicyclic heterocycle, or a spiro heterocycle. A monocyclic heterocycle is a three-, four-, five-, six-, seven-, or eight-membered carbocyclic ring wherein at least one carbon atom is replaced by heteroatom independently selected from the group consisting of O, N, and S. A three- or four-membered ring contains zero or one double bond, and one heteroatom selected from the group consisting of O, N, and S. A five-membered ring contains zero or one double bond and one, two, or three heteroatoms selected from the group consisting of O, N, and S. Examples of five-membered heterocyclic rings include those containing in the ring: 1 O; 1 S; 1 N; 2 N; 3 N; 1 S and 1 N; 1 S, and 2 N; 1 O and 1 N; or 1 O and 2 N. Non limiting examples of 5-membered heterocyclic groups include 1,3-dioxolanyl, tetrahydrofuranyl, dihydrofuranyl, tetrahydrothienyl, dihydrothienyl, imidazolidinyl, oxazolidinyl, imidazolinyl, isoxazolidinyl, pyrazolidinyl, pyrazolinyl, pyrrolidinyl, 2-pyrrolinyl, 3-pyrrolinyl, thiazolinyl, and thiazolidinyl. A six-membered ring contains zero, one, or two double bonds and one, two, or three heteroatoms selected from the group consisting of O, N, and S. Examples of six-membered heterocyclic rings include those containing in the ring: 1 O; 2 O; 1 S; 2 S; 1 N; 2 N; 3 N; 1 S, 1 O, and 1 N; 1 S and 1 N; 1 S and 2 N; 1 S and 1 O; 1 S and 2 O; 1 O and 1 N; and 1 O and 2 N. Examples of 6-membered heterocyclic groups include tetrahydropyranyl, dihydropyranyl, dioxanyl, 1,4-dithianyl, hexahydropyrimidine, morpholinyl, piperazinyl, piperidinyl, 1,2,3,6-tetrahydropyridinyl, tetrahydrothiopyranyl, thiomorpholinyl, thioxanyl, and trithianyl. Seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyridinyl, tetrahydropyranyl, tetrahydrothienyl, thiadiazolinyl, thiadiazolidinyl, thiazolinyl, thiazolidinyl, thiomorpholinyl, thiopyranyl, and trithianyl. The bicyclic heterocycle is a monocyclic heterocycle fused to a phenyl group, or a monocyclic heterocycle fused to a C3-C6 cycloalkyl, or a monocyclic heterocycle fused to a C4-C6 cycloalkenyl, or a monocyclic heterocycle fused to a monocyclic heterocycle. Representative examples of bicyclic heterocycles include, but are not limited to, benzopyranyl, benzothiopyranyl, 2,3-dihydrobenzofuranyl, 2,3-dihydrobenzothienyl, 2,3-dihydro-1H-indolyl, 3,4-dihydroisoquinolin-2(1H)-yl, 2,3,4,6-tetrahydro-1H-pyrido[1,2-a]pyrazin-2-yl, hexahydropyrano[3,4-b][1,4]oxazin-1 (5H)-yl, hexahydropyrrolo[3,4-c]pyrrol-2(1H)-yl, and hexahydrocyclopenta[c]pyrrol-3a(1H)-yl. The monocyclic heterocycle and the bicyclic heterocycle may further contain one or two alkylene bridges, each consisting of 1, 2, 3, or 4 carbon atoms and each linking two non-adjacent atoms of the ring system. Examples of such bridged heterocycles include, but are not limited to, azabicyclo[2.2.1]heptyl (including 2-azabicyclo[2.2.1]hept-2-yl), 8-azabicyclo[3.2.1]oct-8-yl, octahydro-2,5-epoxypentalene, hexahydro-2H-2,5-methanocyclopenta[b]furan, hexahydro-1H-1,4-methanocyclopenta[c]furan, aza-admantane (1 azatricyclo[3.3.1.13,7]decane), and oxa-adamantane (2-oxatricyclo[3.3.1.13,7]decane). The term “spiro heterocycle” as used herein, means a monocyclic heterocycle as defined herein wherein two substituents on the same carbon atom of the monocyclic heterocycle ring together with said carbon atom form a second monocyclic heterocycle or a C3-C6 cycloalkyl ring. Non limiting examples of the spiro heterocycle include 6-azaspiro[3.4]octane, 2-oxa-6-azaspiro[3.4]octan-6-yl, and 2,7-diazaspiro[4.4]nonane. The monocyclic, the bicyclic, and the spiro heterocycles, including exemplary rings, are optionally substituted unless otherwise indicated. The monocyclic, the bicyclic, and the spiro heterocycles are connected to the parent molecular moiety through any carbon atom or any nitrogen atom contained within the ring systems. The nitrogen and sulfur heteroatoms in the heterocycle rings may optionally be oxidized (e.g. 1,1-dioxidotetrahydrothienyl, 1,1-dioxido-1,2-thiazolidinyl, 1,1-dioxidothiomorpholinyl)) and the nitrogen atoms may optionally be quartemized.

The term “4-6 membered monocyclic heterocycle” or “4-6 membered monocyclic heterocyclic” as used herein, means a 4-, 5-, or 6-membered monocyclic heterocycle as defined herein above. Examples of 4-6 membered monocyclic heterocycle include azetidinyl, dihydropyranyl, pyrrolidinyl, tetrahydrofuranyl, tetrahydropyranyl, piperazinyl, piperidinyl, thiomorpholinyl, and morpholinyl. The 4-6 membered monocyclic heterocycles, including exemplary rings, are optionally substituted unless indicated otherwise.

The term “monocyclic heteroaryl” as used herein, means a 5- or 6-membered monocyclic aromatic ring. The five-membered ring contains two double bonds. The five membered ring may contain one heteroatom selected from the group consisting of O and S; or one, two, three, or four nitrogen atoms and optionally one oxygen or one sulfur atom. The six-membered ring contains three double bonds and one, two, three, or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The monocyclic heteroaryls, including exemplary rings, are optionally substituted unless otherwise indicated. The monocyclic heteroaryls are connected to the parent molecular moiety through any substitutable carbon atom or any substitutable nitrogen atom contained within the ring systems. The nitrogen atom in the heteroaryl rings may optionally be oxidized and may optionally be quarternized.

The term “heteroatom” as used herein, means a nitrogen, oxygen, and sulfur.

The term “oxo” as used herein, means a ═O group.

The term “radiolabel” means a compound of the invention in which at least one of the atoms is a radioactive atom or a radioactive isotope, wherein the radioactive atom or isotope spontaneously emits gamma rays or energetic particles, for example alpha particles or beta particles, or positrons. Examples of such radioactive atoms include, but are not limited to, 3H (tritium), 14C, 11C, 15O, 18F, 35S, 123I, and 125I.

A moiety is described as “substituted” when a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

The terms “treat”, “treating”, and “treatment” refer to a method of alleviating or abrogating a disease and/or its attendant symptoms. In certain embodiments, “treat,” “treating,” and “treatment” refer to ameliorating at least one physical parameter, which may not be discernible by the subject. In yet another embodiment, “treat”, “treating”, and “treatment” refer to modulating the disease or disorder, either physically (for example, stabilization of a discernible symptom), physiologically (for example, stabilization of a physical parameter), or both. In a further embodiment, “treat”, “treating”, and “treatment” refer to slowing the progression of the disease or disorder.

The phrase “therapeutically effective amount” means an amount of a compound, or a pharmaceutically acceptable salt thereof, sufficient to prevent the development of or to alleviate to some extent one or more of the symptoms of the condition or disorder being treated when administered alone or in conjunction with another therapeutic agent for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

The term “subject” is defined herein to refer to animals such as mammals, including, but not limited to, primates (e.g., humans), cows, sheep, goats, pigs, horses, dogs, cats, rabbits, rats, mice and the like. In one embodiment, the subject is a human. The terms “human,” “patient,” and “subject” are used interchangeably herein.

The term ‘one or more’ refers to one to four. In one embodiment it refers to one or three. In another embodiment it refers to one to three. In a further embodiment it refers to one to two. In yet other embodiment it refers to two. In yet other further embodiment it refers to one.

As used herein “CF” refers to cystic fibrosis (also known as mucoviscidosis).

As used herein “CFTR” refers to the Cystic Fibrosis Transmembrane Conductance Regulator. In particular embodiment the CFTR is mammalian CFTR, more specifically, human CFTR, a 1480 amino acid protein. The sequence of human CFTR is provided under accession number P13569.

As used herein “wild type CFTR” refers to a native or non-mutant sequence, typically a protein sequence. Wild type CFTR refers to native CFTR, and particularly native mammalian CFTR (mCFTR) or human CFTR (hCFTR) that has normal chloride channel activity in a membrane. “Wild type CFTR sequence” herein refers to the native primary amino acid sequence. More specifically the term “wild type CFTR” refers to a protein having an amino acid sequence according to SEQ ID NO: 1.

As used herein, “class I mutation(s)” refers to mutations which interfere with protein synthesis. They result in the introduction of a premature signal of termination of translation (stop codon) in the mRNA. The truncated CFTR proteins are unstable and rapidly degraded, so, the net effect is that there is no protein at the apical membrane. In particular, Class I mutation(s) refers to mutations between positions 1164 and 1480 of the CFTR protein. More specifically, class I mutation(s) refers to W1282X mutation.

### Potentiators and Correctors

“P potentiator” or “P” as used herein refers to any suitable modulator of the function of CFTR protein. In particular, the P potentiators exhibit improvement in channel activity of a mutant CFTR protein. In particular embodiments of the invention P potentiator is selected from compounds of formula (I) and formula (II). The compounds of formula (I) and formula (II), and methods of making and use of the same, are disclosed in WO2015/018823 and U.S. patent application Ser. No. 15/164,317, the entire disclosure being incorporated herein by reference.

Compounds of formula (I) are as shown below:

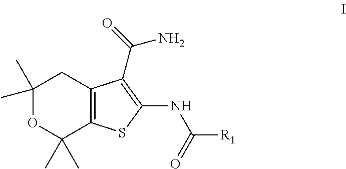

wherein

R¹ is
C₃₋₇ mono or spirocyclic cycloalkyl, optionally substituted with one or more independently selected R² groups,
4-7 membered mono or spirocyclic heterocycloalkyl comprising one or more heteroatoms independently selected from O, N, and S, substituted with one or more independently selected R² groups,
C₆₋₁₀ monocyclic or bicyclic aryl optionally substituted with one or more independently selected R³ groups,
5-10 membered monocyclic or fused bicyclic heteroaryl comprising one or more heteroatoms independently selected from N, O, and S, and optionally substituted with one or more independently selected R³ groups, or
C₁₋₆ alkyl optionally substituted with one or more independently selected R⁴ groups,

each R² is selected from
halo,
OH,
—CN,
—OC(═O)C₁₋₄ alkyl.
—C(═O)—C₁₋₄ alkoxy,
oxo,
C₁₋₄ alkyl (optionally substituted with one or more independently selected R^{5a}), and
C₁₋₄ alkoxy (optionally substituted with one or more independently selected R^{5a}),

each R³ is selected from
halo,
—OH,
—CN,
C₁₋₄ alkyl (optionally substituted with one or more independently selected R^{5b}),
C₁₋₄ alkoxy (optionally substituted with one or more independently selected R^{5b}),
C₂₋₄ alkenyl (optionally substituted with one or more independently selected R^{5b}),
C₃₋₇ monocyclic cycloalkyl,
4-7 membered monocyclic heterocycloalkyl comprising one or more heteroatoms independently selected from N, O, and S,
4-7 membered monocyclic heterocycloalkenyl comprising one or more heteroatoms independently selected from N, O, and S,
5-10 membered monocyclic or fused bicyclic heteroaryl comprising one or more heteroatoms independently selected from N, O, and S, and
—NHSO₂—C₁₋₄ alkyl;

each R⁴ is selected from
halo,
OH,
C₃₋₇ monocyclic cycloalkyl,
—CN, and
C₁₋₄ alkoxy (optionally substituted with one or more independently selected R^{5c}),

each R^{5a}, R^{5b}, and R^{5c} is independently selected from
halo,
OH,
—OP(═O)₂OH,
—CN,
—NR^{6a}R^{6b}, and
C₁₋₄ alkoxy; and
   each R^{6a}, or R^{6b} is independently selected from H, and C₁₋₄ alkyl.

Compounds of formula (II) are as shown below

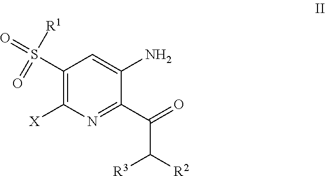

wherein
X is
   H;
   halo;
   C₁₋₄ alkyl optionally substituted with one or more independently selected halo;
   C₁₋₄ alkoxy optionally substituted with one or more independently selected —OH;
      C₁₋₄ alkoxy; or
      —NR^{11A}R^{11B};
   —NR^{12A}R^{12B};
   cyclopropyl optionally substituted with one or more independently selected R⁵ groups;
   phenoxy optionally substituted with one or more independently selected R⁵ groups; or
   phenyl optionally substituted with one or more independently selected R⁵ groups;
R¹ is
   C₁₋₄ alkyl optionally substituted with one or more independently selected —OH;
      C₁₋₄ alkoxy; or
      4-6 membered monocyclic heterocycle comprising 1 or 2 heteroatoms independently selected from the group consisting of O, S, and N;
   phenyl optionally substituted with one or more independently selected R⁴ groups;
   N-linked 4-6 membered monocyclic heterocycle comprising 1, 2, or 3 heteroatoms independently selected from the group consisting of N, O, and S, wherein the monocyclic heterocycle is optionally substituted with one or more independently selected R⁵ groups;
   N-linked 4-6 membered monocyclic heterocycle comprising 1, 2, or 3 heteroatoms independently selected from the group consisting of N, O, and S, fused to a phenyl, wherein the monocyclic heterocycle and the phenyl are optionally substituted with one or more independently selected R⁵ groups;
   C₃₋₇ cycloalkyl optionally substituted with one or more independently selected R⁵ groups; or
   —NR⁶R⁷;
R² is
   H;
   C₁₋₆ alkyl optionally substituted with one or more independently selected —OH;
      halo;
      C₁₋₄ alkoxy optionally substituted with one or more independently selected halo;
         C₁₋₄ alkoxy;
         C₃₋₇ cycloalkyl optionally substituted with one or more independently selected R⁵ groups; or
         4-6 membered monocyclic heterocycle comprising 1 or 2 heteroatoms independently selected from the group consisting of N, O, and S, wherein the monocyclic heterocycle is optionally substituted with one or more independently selected R⁵ groups;
      —C(═O)NR^{8a}R^{8b};
      C₃₋₇ cycloalkyl optionally substituted with one or more independently selected —OH;
         halo;
         C₁₋₄ alkoxy optionally substituted with one or more independently selected halo; or
         C₁₋₄ alkyl optionally substituted with one or more independently selected —OH, halo, or C₁₋₄ alkoxy;
      4-6 membered monocyclic heterocycle comprising 1 or 2 heteroatoms independently selected from the group consisting of N, O, and S, wherein the monocyclic heterocycle is optionally substituted with one or more independently selected —OH;
         halo;
         C₁₋₄ alkoxy optionally substituted with one or more independently selected halo, or
         C₁₋₄ alkyl optionally substituted with one or more independently selected halo;
      5-6 membered monocyclic heteroaryl comprising 1, 2, or 3 heteroatoms independently selected from the group consisting of O, S, and N, wherein the monocyclic heteroaryl is optionally substituted with one or more independently selected R⁵ groups; or
      phenyl optionally substituted with one or more independently selected R⁵ groups;
   C₃₋₇ cycloalkyl optionally substituted with one or more —OH;
      halo;
      C₁₋₄ alkyl optionally substituted with one or more independently selected halo or —OH; or
      C₁₋₄ alkoxy optionally substituted with one or more independently selected halo;
   4-6 membered monocyclic heterocycle comprising 1 or 2 heteroatoms independently selected from the group consisting of O, S, and N, wherein the monocyclic heterocycle is optionally substituted with one or more —OH;
      halo;
      C₁₋₄ alkyl optionally substituted with one or more independently selected halo; or
      C₁₋₄ alkoxy optionally substituted with one or more independently selected halo;
   4-6 membered monocyclic heterocycle comprising 1 or 2 heteroatoms independently selected from the group consisting of O, S, and N, fused to a phenyl ring, wherein the monocyclic heterocycle and the phenyl are optionally substituted with one or more independently selected R⁵ groups;
   5-11 membered spirocyclic heterocycle comprising 1 or 2 heteroatoms independently selected from the group consisting of O, S, and N, wherein the spirocyclic heterocycle is optionally substituted with one or more independently selected R⁵ groups;
   5-6 membered monocyclic heteroaryl comprising 1, 2, or 3 heteroatoms independently selected from the group consisting of O, S, and N, wherein the monocyclic heteroaryl is optionally substituted with one or more independently selected R⁵ groups; or
   —NHC(═O)R¹³;
and R³ is H; or
R² and R³, together with the nitrogen atom to which they are attached form
   an azetidine or a pyrrolidine ring, wherein the azetidine and the pyrrolidine are optionally substituted with one or more independently selected R⁹ groups; or
   a 7-11 membered spirocyclic heterocycle comprising one or more heteroatoms independently selected from the group consisting of N, O, and S; wherein the spirocyclic heterocycle is optionally substituted with one or more independently selected R⁵ groups;
each R⁴ is independently selected from the group consisting of:
   halo;
   C₁₋₄ alkyl optionally substituted with one or more independently selected halo; and
   C₁₋₄ alkoxy optionally substituted with one or more independently selected halo;
each R⁵ is independently selected from the group consisting of:
   —OH;
   halo;
   C₁₋₄ alkyl optionally substituted with one or more independently selected
      C₁₋₄ alkoxy;
      halo; or
      —OH; and
   C₁₋₄ alkoxy optionally substituted with one or more independently selected halo;
R⁶ is H, C₁₋₄ alkyl, or C₃₋₇ cycloalkyl wherein the C₃₋₇ cycloalkyl is optionally substituted with one or more independently selected R⁵ groups;
R⁷ is
   C₁₋₄ alkyl optionally substituted with one or more independently selected halo;
      phenyl optionally substituted with one or more independently selected
         halo;
         C₁₋₄ alkyl optionally substituted with one or more independently selected halo; or
         C₁₋₄ alkoxy optionally substituted with one or more independently selected halo;
      C₁₋₄ alkoxy optionally substituted with one or more independently selected halo; or
      4-6 membered monocyclic heterocycle comprising 1 or 2 heteroatoms independently selected from the group consisting of O, S, and N; wherein the monocyclic heterocycle is optionally substituted with one or more independently selected R⁵ groups;
each R^{8a} and R^{8b} is independently selected from the group consisting of H;
   C₁₋₄ alkyl optionally substituted with one or more independently selected halo; and
   C₃₋₇ cycloalkyl optionally substituted with one or more independently selected R⁵ groups;
each R⁹ is independently selected from the group consisting of:
   —OH;
   halo;
   —CN;
   C₁₋₄ alkyl optionally substituted with one or more independently selected —OH;
      halo; or
      C₁₋₄ alkoxy;
   C₁₋₄ alkoxy optionally substituted with one or more independently selected halo;
   C₃₋₇ cycloalkyl optionally substituted with one or more independently selected R⁵ groups; —C(═O)NR^{10a}R^{10b}; and
   4-6 membered monocyclic heterocycle comprising 1 or 2 heteroatoms independently selected from the group consisting of O, S, and N, wherein the monocyclic heterocycle is optionally substituted with one or more independently selected R⁵ groups;
each R^{10a} and R^{10b} is independently selected from the group consisting of H and C₁₋₄ alkyl;
each R^{11a} and R^{11b} is independently selected from the group consisting of H; and
   C₁₋₄ alkyl;
R^{12a} and R^{12b} are independently selected from the group consisting of H;
   C₁₋₄ alkyl; and
   C₃₋₇ cycloalkyl; and
R¹³ is independently C₁₋₄ alkyl optionally substituted with one or more independently selected —OH;
   halo; or
C₁₋₄ alkoxy.

In a more specific embodiment P potentiator is a compound of formula

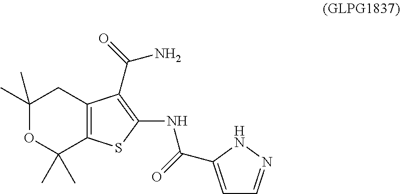 [SMILES: CC1(C)Cc2c(sc(NC(=O)c3ccn[nH]3)c2C(N)=O)C(C)(C)O1]

As used herein the term C corrector refers to any corrector molecule that is not a read-through corrector. The term “read-through correctors” as used herein refers to any molecule that acts on RNA level to allow read-through of premature termination codon (PTC). In particular C corrector can be either C1 corrector or C2 corrector as defined herein.

As used herein the term “C1 corrector” or “C1” refers to refers to a modulator of the cellular processing and/or localization. More specifically C1 corrector is not a read-through corrector. In particular embodiment C1 corrector is selected from compounds of formula (III). The compounds of formula (III), and methods of making and use of the same, are disclosed in U.S. patent application Ser. No. 14/925,649, the entire disclosure being incorporated herein by reference.

The compounds of formula (III) are as show below:

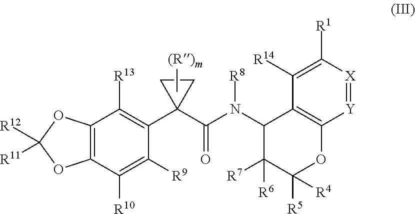

wherein
X is CR² and Y is CR³; or
X is N and Y is CR³; or
X is CR² and Y is N;
m is 0, 1, 2, or 3;
R″ are optional substituents on the cyclopropyl ring, and at each occurrence, are each independently halogen, C₁-C₆ haloalkyl, or C₁-C₆ alkyl;
R¹ and R², are each independently hydrogen, halogen, C₁-C₆ haloalkyl, C₁-C₆ alkyl, —OR^{1A}, —C(O)OR^{1B}, —NR^{1A}R^{2A}, or —C(O)NR^{1A}R^{2A};
R^{1A} and R^{2A}, at each occurrence, are each independently hydrogen, C₁-C₆ haloalkyl,
G^{1A}, or C₁-C₆ alkyl; wherein the C₁-C₆ haloalkyl and the C₁-C₆ alkyl are each optionally substituted with one or two substituents independently selected from the group consisting
of —OR^{ZA}, —SR^{ZA}, —S(O)₂R^{ZA}, —C(O)R^{ZA}, —C(O)OR^{ZA}, —C(O)N(R^{ZA})₂, —N(R^{ZA})₂, —N(R^{ZA})C (O)R^{ZB}, —N(R^{ZA})S(O)₂R^{ZB}, —N(R^{ZA})C(O)OR^{ZB}, —N(R^{ZA})C(O)N(R^{ZA})₂, —CN, and G^{1A}; or
R^{1A} and R^{2A} together with the nitrogen atom to which they are attached form a 4-6 membered heterocycle wherein the 4-6 membered heterocycle is optionally substituted with 1, 2, or 3 substituents independently selected from the group consisting of halogen, C₁-C₆ alkyl, C₁-C₆ haloalkyl, —OR^{j}, and N(R^{j})₂; wherein
R^{ZA}, at each occurrence, is independently hydrogen, C₁-C₆ haloalkyl, C₁-C₆ alkyl, G^{1A}, or —(C₁-C₆ alkylenyl)-G^{1A}; and
R^{ZB}, at each occurrence, is independently C₁-C₆ haloalkyl, C₁-C₆ alkyl, G^{1A}, or —(C₁-C₆ alkylenyl)-G^{1A};
R^{1B} is hydrogen, C₁-C₆ haloalkyl, or C₁-C₆ alkyl;
R³ and R¹⁴, are each independently hydrogen, halogen, C₁-C₆ haloalkyl, C₁-C₆ alkyl, —OH, or —O—(C₁-C₆ alkyl);
R⁴ is hydrogen, C₁-C₆ haloalkyl, or C₁-C₆ alkyl;
R⁵ is hydrogen, —C(O)R¹, —C(O)OH, —C(O)O(C₁-C₆ alkyl), —C(O)N(R^{h})₂, C₁-C₆ haloalkyl, C₁-C₆ alkyl, or G^{2A}; wherein the C₁-C₆ haloalkyl and the C₁-C₆ alkyl are each optionally substituted with one or two substituents independently selected from the group consisting
of —OR^{h}, —OC(O)N(R^{h})₂, —C(O)R^{h}, —C(O)OR^{h}, —C(O)N(R^{h})₂, —N(R^{h})₂, —N(R^{h})C(O)Rⁱ, —N(R^{h})S(O)₂Rⁱ, —N(R^{h})C(O)O(Rⁱ), —N(R^{h})C(O)N(R^{h})₂, and G^{2A}; or
R⁴ and R⁵, together with the carbon atom to which they are attached, form a C₃-C₆ cycloalkyl or a 4-6 membered heterocycle; wherein the C₃-C₆ cycloalkyl and the 4-6 membered heterocycle are each optionally substituted with 1, 2, or 3 independently selected R^{p} groups;
G^{2A}, at each occurrence, is independently cycloalkyl, cycloalkenyl, heterocycle, aryl, or heteroaryl, each of which is independently unsubstituted or substituted with 1, 2, or 3 independently selected R^{q} groups;
R^{p} and R^{q}, at each occurrence, are each independently C₁-C₆ alkyl, halogen, C₁-C₆ haloalkyl, —CN, oxo,
NO₂, —OR^{h}, —OC(O)Rⁱ, —OC(O)N(R^{h})₂, —SR^{h}, —S(O)₂R^{h}, —S(O)₂N(R^{h})₂, —C(O)R^{h}, —C(O)OR^{h}, —C(O)N(R^{h})₂, —C(O)N(R^{h})S(O)₂R^{h}, —N(R^{h})₂, —N(R^{h})C(O)R, —N(R^{h})S(O)₂Rⁱ, —N(R^{h})C(O)O(Rⁱ), —N(R^{h})C(O)N(R^{h})₂, or G^{A}, wherein the C₁-C₆ haloalkyl and the C₁-C₆ alkyl are each optionally substituted with one or two substituents independently selected from the group consisting
of —OR^{h}, —OC(O)Rᵢ, —OC(O)N(R^{h})₂, —SR^{h}, —S(O)₂R^{h}, —S(O)₂N(R^{h})₂, —C(O)R^{h}, —C(O)OR^{h}, —C(O)N(R^{h})₂, —C(O)N(R^{h})S(O)₂R^{h}, —N(R^{h})₂, —N(R^{h})C(O)Rⁱ, —N(R^{h})S(O)₂Rⁱ, —N(R^{h})C(O)O (Rⁱ), —N(R^{h})C(O)N(R^{h})₂, —CN, and G^{A};
R^{h}, at each occurrence, is independently hydrogen, C₁-C₆ haloalkyl, C₁-C₆ alkyl, or G^{A}, wherein the C₁-C₆ haloalkyl and the C₁-C₆ alkyl are each optionally substituted with one or two substituents independently selected from the group consisting
of —OR^{j}, —OC(O)N(R^{j})₂, —SR^{j}, —C(O)OR^{j}, —C(O)N(R^{j})₂, —N(R^{j})₂, —CN, and G^{A};
Rⁱ, at each occurrence, is independently C₁-C₆ haloalkyl, C₁-C₆ alkyl, or G^{A}, wherein the C₁-C₆ haloalkyl and the C₁-C₆ alkyl are each optionally substituted with one or two substituents independently selected from the group consisting
of —OR^{j}, —OC(O)N(R^{j})₂, —SR^{j}, —C(O)OR^{j}, —C(O)N(R^{j})₂, —N(R^{j})₂, —CN, and G^{A};
R⁶ is hydrogen, halogen, C₁-C₆ haloalkyl, or C₁-C₆ alkyl;
R⁷ is hydrogen, halogen, —OR, —N(R^{j})₂, —N(R^{j})C(O)R^{k}, C₁-C₆ haloalkyl, C₁-C₆ alkyl, C₂-C₆ alkenyl, or —(C₁-C₆ alkylenyl)-G^{3A};
R⁸ is hydrogen, C₁-C₆ haloalkyl, or C₁-C₆ alkyl;
R⁹, R¹⁰, and R¹³, are each independently hydrogen, halogen, —OR, C₁-C₆ haloalkyl, or C₁-C₆ alkyl;
R¹¹ and R¹² are each independently hydrogen, C₁-C₃ alkyl, or halogen;
G^{1A}, G^{3A}, and G^{A}, at each occurrence, are each independently cycloalkyl, cycloalkenyl, heterocycle, aryl, or heteroaryl, each of which is independently unsubstituted or substituted with 1, 2, or 3 independently selected R^{s} groups; wherein
R^{s}, at each occurrence, is independently C₁-C₆ alkyl, C₂-C₆ alkenyl, C₂-C₆ alkynyl, halogen, C₁-C₆ haloalkyl, —CN, oxo,
NO₂, —OR^{j}, —OC(O)R^{k}, —OC(O)N(R^{j})₂, —SR^{j}, —S(O)₂R^{j}, —S(O)₂N(R^{j})₂, —C(O)R^{j}, —C(O)OR^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, —(C₁-C₆ alkylenyl)-OR, —(C₁-C₆ alkylenyl)-OC(O)R^{k}, —(C₁-C₆ alkylenyl)-OC(O)N(R^{j})₂, —(C₁-C₆ alkylenyl)-SR^{j}, —(C₁-C₆ alkylenyl)-S(O)₂R^{j}, —(C₁-C₆ alkylenyl)-S(O)₂N(R^{j})₂, —(C₁-C₆ alkylenyl)-C(O)R^{j}, —(C₁-C₆ alkylenyl)-C(O)OR^{j}, —(C₁-C₆ alkylenyl)-C(O)N(R^{j})₂, —(C₁-C₆ alkylenyl)-N(R^{j})₂, —(C₁-C₆ alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, or —(C₁-C₆ alkylenyl)-CN;
R^{j}, at each occurrence, is independently hydrogen, C₁-C₆ alkyl, or C₁-C₆ haloalkyl; and
R^{k}, at each occurrence, is independently C₁-C₆ alkyl or C₁-C₆ haloalkyl.

The term “C2 corrector” or “C2” as used herein refers to a modulator of the cellular processing and/or localization. More specifically C2 corrector is not a read-through corrector.

In a particular embodiment of the invention C2 corrector is a compound of formula (IV) or formula (V). The compounds of formula (IV) and formula (V), and methods of making and use of the same, are disclosed in U.S. patent application Ser. No. 15/287,911 and U.S. patent application Ser. No. 15/287,922 respectively, the entire disclosure being incorporated herein by reference.

In some embodiments, the C2 corrector is a compound of formula (IV) or a pharmaceutically acceptable salt thereof,

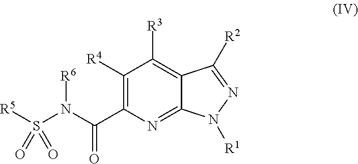

wherein
R¹ is G^{1A}, C₁-C₆ haloalkyl, or C₁-C₆ alkyl; wherein the C₁-C₆ haloalkyl and the C₁-C₆ alkyl are each optionally substituted with one G^{1A};
G^{1A}, at each occurrence, is independently phenyl, 5-6 membered monocyclic heteroaryl, 4-7 membered monocyclic heterocycle, 5-11 membered fused bicyclic heterocycle, or C₃-C₆ monocyclic cycloalkyl; wherein each G^{1A} is optionally substituted with 1, 2, 3, or 4 substituents independently selected from the group consisting of R^{1a} and G^{1B};
G^{1B}, at each occurrence, is independently 4-7 membered monocyclic heterocycle which is optionally substituted with 1, 2, 3, or 4 independently selected R^{1b} groups;
R² is hydrogen, C₂-C₄ alkenyl, C₁-C₆ alkyl, C₁-C₆ haloalkyl, —OR^{2xa}, —N(R^{2xa})(R^{2xb}), or G^{2A};
R^{2xa}, at each occurrence, is independently C₁-C₆ alkyl, C₁-C₆ haloalkyl, or G^{2B};
R^{2xb} is hydrogen, C₁-C₃ alkyl, or C₁-C₃ haloalkyl;
G^{2A} and G^{2B} are each independently a 4-7 membered monocyclic heterocycle or a C₃-C₆ monocyclic cycloalkyl; wherein G^{2A} and G^{2B} are each optionally substituted with 1, 2, or 3 independently selected R^{2a} groups;
R³ is G^{3A}, -G^{3B}-L -G^{3C}, -G^{3B}-L³-G^{3C}-G^{3E}, —(C₁-C₆ alkylenyl)-G³D, —OR^{3a}, or —N(R^{3a})(R^{3b});
R^{3a}, at each occurrence, is independently G^{3D}, C₁-C₆ haloalkyl, or C₁-C₆ alkyl; wherein the C₁-C₆ haloalkyl and the C₁-C₆ alkyl are each optionally substituted with one or two substituents independently selected from the group consisting of G^{3D}, —OR^{3xa}, and —N(R^{3xb})₂;
R^{3xa} and R^{3xb}, at each occurrence, are each independently hydrogen, C₁-C₆ haloalkyl, C₁-C₆ alkyl, or G^{3D};
R^{3b} is hydrogen, C₁-C₆ alkyl, or C₁-C₆ haloalkyl;
L¹ is a bond, C₁-C₆ alkylenyl, (C₁-C₆ alkylenyl)ᵣ-L²-(C₁-C₆ alkylenyl), or O—(C₁-C₆ alkylenyl)-C(O), wherein the left end of the L moiety is attached to G^{3B};
L² is O, N(R^{x}), C(O), N(R^{x})C(O), or C(O)N(R^{x}); wherein each R^{x} is independently hydrogen, C₁-C₆ alkyl, or C₁-C₆ haloalkyl;
L³ is a bond or C₁-C₆ alkylenyl;
r is 0 or 1;
s is 0 or 1;
G^{3A}, G^{3B}, and G^{3C} and each independently C₃-C₁₁ cycloalkyl, phenyl, 5-6 membered monocyclic heteroaryl, or 4-11 membered heterocycle; wherein G^{3A}, G^{3B}, and G^{3C} are each optionally substituted with 1, 2, 3, or 4 independently selected R^{e} groups;
G^{3D}, at each occurrence, is independently C₃-C₈ monocyclic cycloalkyl, 4-7 membered monocyclic heterocycle, a 5-11 membered fused bicyclic heterocycle, or a 5-11 membered spiro heterocycle; wherein each G^{3D} is optionally substituted with 1, 2, 3, or 4 substituents independently selected from the group consisting of R^{e} and G^{3E};
G^{3E}, at each occurrence, is independently C₃-C₈ monocyclic cycloalkyl or 4-7 membered monocyclic heterocycle; wherein each G^{3E} is optionally substituted with 1, 2, 3, or 4 independently selected R^{e} groups;
R⁴ is hydrogen, C₁-C₃ alkyl, or C₁-C₃ haloalkyl;
R⁵ is C₁-C₆ alkyl, C₂-C₆ alkenyl, C₂-C₆ alkynyl, C₁-C₆ haloalkyl, —N(R^{5ax})(R^{5bx}), —OR^{5dx}, or G^{5A};
wherein the C₁-C₆ alkyl and the C₁-C₆ haloalkyl are each optionally substituted with one or two substituents independently selected from the group consisting of
G^{5A}, —CN, —N₃, —OR^{5ax}, —S(O)₂R^{5ax}, —S(O)₂N(R^{5ax})(R^{5bx}), —N(R^{5ax})(R^{5bx}), —N(R^{5bx})S(O)₂R^{5cx}, —N(R^{5bx})C(O)R^{5cx}, —N(R^{5bx})C(O)N(R^{5ax})(R^{5bx}), —N(R^{5bx})C(O)OR^{5cx}, —C(O)R^{5ax}, —C(O)OR^{5ax}, —C(O)N(R^{5bx}) S(O)₂R^{5cx}, and —C(O)N(R^{5ax})(R^{5bx});
R^{5ax} and R^{5bx}, at each occurrence, are each independently hydrogen, C₁-C₆ alkyl, C₁-C₆ haloalkyl, —OR^{5ex}, —(C₁-C₆ alkylenyl)-OR^{5ex}, G^{5A}, or —(C₁-C₆ alkylenyl)-G^{5A};
R^{5cx}, at each occurrence, is independently C₁-C₆ alkyl, C₁-C₆ haloalkyl, G^{5A}, or —(C₁-C₆ alkylenyl)-G^{5A};
R^{5dx} is C₁-C₆ alkyl, or C₁-C₆ haloalkyl;
R^{5ex} is hydrogen, C₁-C₆ alkyl, or C₁-C₆ haloalkyl;
G^{5A}, at each occurrence, is independently C₃-C₁₁ cycloalkyl, phenyl, 5-6 membered monocyclic heteroaryl, or 4-11 membered heterocycle; wherein each G^{5A} is optionally substituted with 1, 2, 3, or 4 independently selected R^{5a} groups;
R^{5a}, at each occurrence, is independently C₁-C₆ alkyl, C₂-C₆ alkenyl, C₂-C₆ alkynyl, halogen, C₁-C₆ haloalkyl, oxo, G^{5B}, —CN, NO₂, —OR^{b}, —OC(O)R^{c}, —OC(O)N(R^{d})₂, —SR^{b}, —S(O)₂R^{b}, —S(O)₂N(R^{d})₂, —C(O)R^{b}, —C(O)OR^{b}, —C(O)N (R^{d})₂, —C(O)N(R^{d})S(O)₂R^{c}, —N(R^{d})₂, —N(R^{d})C(O)R^{c}, —N(R^{d})S(O)₂R^{c}, —N(R^{d})C(O)O(R^{b}), —N(R^{d})C(O)N(R^{d})₂, —N(R^{d})S(O)₂N(R^{d})₂, —(C₁-C₆ alkylenyl)-CN, —(C₁-C₆ alkylenyl)-G^{5B}, —(C₁-C₆ alkylenyl)-OR^{b}, —(C₁-C₆ alkylenyl)-OC(O)R^{c}, —(C₁-C₆ alkylenyl)-OC(O)N(R^{d})₂, —(C₁-C₆ alkylenyl)-SR^{b}, —(C₁-C₆ alkylenyl)-S(O)₂R^{b}, —(C₁-C₆ alkylenyl)-S(O)₂N(R^{d})₂, —(C₁-C₆ alkylenyl)-C(O)R^{b}, —(C₁-C₆ alkylenyl)-C(O)OR^{b}, —(C₁-C₆ alkylenyl)-C(O)N(R^{d})₂, —(C₁-C₆ alkylenyl)-C(O)N(R^{d})S(O)₂R^{c}, —(C₁-C₆ alkylenyl)-N(R^{d})₂, —(C₁-C₆ alkylenyl)-N(R^{d})C(O)R^{c}, —(C₁-C₆ alkylenyl)-N(R^{d})S(O)₂R^{c}, —(C₁-C₆ alkylenyl)-N(R^{d})C(O)O(R^{c}), —(C₁-C₆ alkylenyl)-N(R^{d})C(O)N(R^{d})₂, or —(C₁-C₆ alkylenyl)-N(R^{d})S(O)₂N(R^{d})₂;
R^{b} and R^{d}, at each occurrence, are each independently hydrogen, C₁-C₆ alkyl, C₁-C₆ haloalkyl, alkoxyalkyl, G^{5B}, or —(C₁-C₆ alkylenyl)-G^{5B};
R^{c}, at each occurrence, is independently C₁-C₆ alkyl, C₁-C₆ haloalkyl, alkoxyalkyl, G^{5B}, or —(C₁-C₆ alkylenyl)-G^{5B};
G^{5B}, at each occurrence, is independently C₃-C₆ monocyclic cycloalkyl, phenyl, 5-6 membered monocyclic heteroaryl, or 4-7 membered monocyclic heterocycle; wherein each G^{5B} is optionally substituted with 1, 2, 3, or 4 independently selected R^{5b} groups;
R^{e}, at each occurrence, is independently C₂-C₆ alkenyl, C₂-C₆ alkynyl, C₁-C₆ alkyl, C₁-C₆ haloalkyl, halogen, oxo, —CN, —N₃, NO₂, —OR, —OC(O)R^{g}, —OC(O)NR^{f}R^{h}, —SR^{f}, —S(O)₂R^{f}, —S(O)₂NR^{f}R^{h}, —C(O)R^{f}, —C(O)OR^{f}, —C(O)NR^{f}R^{h}, —C(O)N(R^{h})S(O)₂R^{f}, —N(R^{f})₂, —N(R^{h})C(O)R^{h}, —N(R^{h})S(O)₂R^{g}, —N(R^{h})C(O)O(R^{g}), —N(R^{h})C(O)N R^{f}R^{h}, or —N(R^{h})S(O)₂NR^{f}R^{h}; wherein the C₁-C₆ haloalkyl and the C₁-C₆ alkyl are each optionally substituted with 1 or 2 substituents independently selected from the group consisting of —CN, NO₂, —OR^{f}, —OC(O)R^{g}, —OC(O)NR^{f}R^{h}, —SR^{f}, —S(O)₂R^{f}, —S(O)₂NR^{f}R^{h}, —C(O)R^{f}, —C(O)OR^{f}, —C(O)NR^{f}R^{h}, —C(O)N(R^{h})S(O)₂R^{f}, —N(R^{f})₂, —N(R^{h})C(O)R^{g}, —N(R^{h})S(O)₂R^{g}, —N(R^{h})C(O)O(R^{g}), —N(R^{h})C(O)N R^{f}R^{h}, and —N(R^{h})S(O)₂NR^{f}R^{h};
R^{f}, at each occurrence, is independently hydrogen, C₁-C₆ alkyl, C₂-C₆ alkenyl, C₂-C₆ alkynyl, C₁-C₆ haloalkyl, —(C₁-C₆ alkylenyl)-CN, —(C₁-C₆ alkylenyl)-OR^{m}, —(C₁-C₆ alkylenyl)-OC(O)R^{m}, —(C₁-C₆ alkylenyl)-OC(O)N(R^{m})₂, —(C₁-C₆ alkylenyl)-SR^{m}, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-C(O)N(R^{m})S(O)₂Rⁿ, —(C₁-C₆ alkylenyl)-N(R^{m})₂, —(C₁-C₆ alkylenyl)-N(R^{m})C(O)Rⁿ, —(C₁-C₆ alkylenyl)-N(R^{m})S(O)₂Rⁿ, —(C₁-C₆ alkylenyl)-N(R^{m})C(O)O(Rⁿ), —(C₁-C₆ alkylenyl)-N(R^{m})C(O)N(R^{m})₂, or —(C₁-C₆ alkylenyl)-N(R^{m})S(O)₂N(R^{m})₂;
R^{g}, at each occurrence, is independently C₁-C₆ alkyl, C₂-C₆ alkenyl, C₂-C₆ alkynyl, C₁-C₆ haloalkyl, —(C₁-C₆ alkylenyl)-CN, —(C₁-C₆ alkylenyl)-OR^{m}, —(C₁-C₆ alkylenyl)-OC(O)R^{m}, —(C₁-C₆ alkylenyl)-OC(O)N(R^{m})₂, —(C₁-C₆ alkylenyl)-SR^{m}, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-C(O)N(R^{m})S(O)₂Rⁿ, —(C₁-C₆ alkylenyl)-N(R^{m})₂, —(C₁-C₆ alkylenyl)-N(R^{m})C(O)Rⁿ, —(C₁-C₆ alkylenyl)-N(R^{m})S(O)₂Rⁿ, —(C₁-C₆ alkylenyl)-N(R^{m})C(O)O(Rⁿ), —(C₁-C₆ alkylenyl)-N(R^{m})C(O)N(R^{m})₂, or —(C₁-C₆ alkylenyl)-N(R^{m})S(O)₂N(R^{m})₂;
R^{h}, at each occurrence, is independently hydrogen, C₁-C₆ alkyl, C₁-C₆ haloalkyl, or —(C₁-C₆ alkylenyl)-OR^{m};
R^{1a}, R^{1b}, R^{2a}, and R^{5b}, at each occurrence, are each independently C₁-C₆ alkyl, C₂-C₆ alkenyl, C₂-C₆ alkynyl, halogen, C₁-C₆ haloalkyl, oxo, —CN, NO₂, —OR^{m}, —OC(O)Rⁿ, —OC(O)N(R^{m})₂, —SR^{m}, —S(O)₂R^{m}, —S(O)₂N(R^{m})₂, —C(O)R^{m}, —C(O)OR^{m}, —C(O)O(benzyl), —C(O)N(R^{m})₂, —C(O)N(R^{m})S(O)₂Rⁿ, —N(R^{m})₂, —N(R^{m})(alkoxyalkyl), —N(alkoxyalkyl)₂, —N(R^{m})C(O)Rⁿ, —N(R^{m})S(O)₂Rⁿ, —N(R^{m})C(O)O(Rⁿ), —N(R^{m})C(O)N(R^{m})₂, —N(R^{m})S(O)₂N(R^{m})₂, —(C₁-C₆ alkylenyl)-CN, —(C₁-C₆ alkylenyl)-OR^{m}, —(C₁-C₆ alkylenyl)-OC(O)Rⁿ, —(C₁-C₆ alkylenyl)-OC(O)N(R^{m})₂, —(C₁-C₆ alkylenyl)-SR^{m}, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-C(O)N(R^{m})S(O)₂R^{m}, —(C₁-C₆ alkylenyl)-N(R^{m})₂, —(C₁-C₆ alkylenyl)-N(R^{m})C(O)Rⁿ, —(C₁-C₆ alkylenyl)-N(R^{m})S(O)₂Rⁿ, —(C₁-C₆ alkylenyl)-N(R^{m})C(O)O(Rⁿ), —(C₁-C₆ alkylenyl)-N(R^{m})C(O)N(Rⁿ)₂, or —(C₁-C₆ alkylenyl)-N(R^{m})S(O)₂N(Rⁿ)₂;
R^{m}, at each occurrence, is independently hydrogen, C₁-C₆ alkyl, or C₁-C₆ haloalkyl;
Rⁿ, at each occurrence, is independently C₁-C₆ alkyl or C₁-C₆ haloalkyl;
R⁶ is hydrogen, C₁-C₆ alkyl, or C₁-C₆ haloalkyl; or
R⁵ and R⁶ together form a C₁-C₆ alkylenyl or —N(R^{z})—(C₁-C₆ alkylenyl)- wherein the N(R^{z}) is attached to the S(O)₂ moiety of formula (I); and
   R^{z} is hydrogen, C₁-C₆ alkyl, or C₁-C₆ haloalkyl.

In some embodiments, the corrector C2 is a compound of formula (V) or a pharmaceutically acceptable salt thereof,

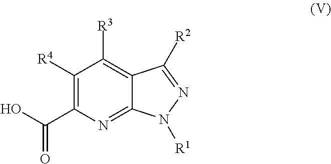

wherein
R¹ is G^{1A}, -G^{1B}-G^{1C}, -G^{1B}-L^{1A}-G^{1C}, C₁-C₆ haloalkyl, C₁-C₆ alkyl, —(C₁-C₆ alkylenyl)-CN, —(C₁-C₆ alkylenyl)-G^{1D}, or -G^{1D}-O-benzyl;
L^{1A} is —O— or —O—(C₁-C₃ alkylenyl)-; wherein the left end of the L^{1A} moiety is attached to G^{1B};
G^{1A} is phenyl, aryl, 5-6 membered monocyclic heteroaryl, 4-7 membered monocyclic heterocycle, fused bicyclic heterocycle, or C₃-C₆ monocyclic cycloalkyl; wherein each G^{1A} is optionally substituted with 1, 2, 3, or 4 independently selected R^{1a} groups;
G^{1B} is phenyl or 5-6 membered monocyclic heteroaryl; wherein each G^{1B} is optionally substituted with 1, 2, 3, or 4 independently selected R^{1b} groups;
G^{1C} is 4-7 membered monocyclic heterocycle which is optionally substituted with 1, 2, 3, or 4 independently selected R^{1c} groups;
G^{1D}, at each occurrence, is a 4-7 membered monocyclic heterocycle, 5-6 membered monocyclic heteroaryl, or a C₃-C₆ monocyclic cycloalkyl; wherein each G^{1D} is optionally substituted with 1, 2, 3, or 4 independently selected R^{1d} groups;
R² is C₂-C₄ alkenyl, C₁-C₆ alkyl, C₁-C₆ haloalkyl, —OR^{2xa}, —(C₁-C₆ alkylenyl)-OR^{2xb}, —(C₁-C₆ alkylenyl)-N(R^{2xb})₂, —C(O)OR^{2xb}, —C(O)N(R^{2xb})₂, or -G^{2A};
R^{2xa} is hydrogen, C₁-C₆ alkyl, C₁-C₆ haloalkyl, or G^{2B};
R^{2xb}, at each occurrence, is independently hydrogen, C₁-C₆ alkyl, or C₁-C₆ haloalkyl;
G^{2A} and G^{2B} are each independently 4-7 membered monocyclic heterocycle or C₃-C₆ monocyclic cycloalkyl; wherein G^{2A} and G^{2B} are each optionally substituted with 1, 2, or 3 independently selected R^{2a} groups;
R³ is halogen, G^{3A}, -G^{3B}-L¹-G^{3C}, -G^{3B}-L³-G^{3C}-L⁴-G^{3F}, —(C₁-C₆ alkylenyl)-G^{3E}, —OR^{3a}, —N(R^{3a})(R^{3b}), —N(R^{3b})C(O)G^{3D}, or —C(O)G^{3D};
R^{3a}, at each occurrence, is independently G^{3E}, C₁-C₆ haloalkyl, or C₁-C₆ alkyl; wherein the C₁-C₆ haloalkyl and the C₁-C₆ alkyl are each optionally substituted with one or two substituents independently selected from the group consisting of G^{3E}, —OR^{3xa}, —C(O)G^{3D}, —N(R^{3xb})₂, and —S(O)₂R^{3xc};
R^{3xa}, R^{3xb}, and R^{3xc}, at each occurrence, are each independently hydrogen, C₁-C₆ haloalkyl, C₁-C₆ alkyl, G^{3E}, —(C₁-C₆ alkylenyl)-OR^{3ya}, or —(C₁-C₆ alkylenyl)-N(R^{3ya})₂; wherein R^{3ya}, at each occurrence, is independently hydrogen, C₁-C₆ alkyl, or C₁-C₆ haloalkyl;
R^{3b}, at each occurrence, is hydrogen, C₁-C₆ alkyl, or C₁-C₆ haloalkyl;
L¹ is a bond, C₁-C₆ alkylenyl, (C₁-C₆ alkylenyl)ᵣ-L²-(C₁-C₆ alkylenyl)ₛ, or O—(C₁-C₆ alkylenyl)-C(O), wherein the left end of the L₁ moiety is attached to G^{3B};
L² is O, N(R^{x}), C(O), N(R^{x})C(O), or C(O)N(R^{x}); wherein each R^{x} is independently hydrogen, C₁-C₆ alkyl, or C₁-C₆ haloalkyl;
L³ is a bond or C₁-C₆ alkylenyl;
L⁴ is a bond, C₁-C₆ alkylenyl, O, N(R^{2x}), C(O), N(R^{2x})C(O), or C(O)N(R^{2x}); wherein each R^{2x} is independently hydrogen, C₁-C₆ alkyl, or C₁-C₆ haloalkyl;
r is 0 or 1;
s is 0 or 1;
G^{3A}, G^{3B}, and G^{3C}, are each independently C₃-C₁₁ cycloalkyl, phenyl, 5-6 membered monocyclic heteroaryl, or 4-11 membered heterocycle, wherein G^{3A}, G^{3B}, and G^{3C} are each optionally substituted with 1, 2, 3, or 4 independently selected R^{e} groups;
G^{3D}, at each occurrence, is 4-7 membered monocyclic heterocycle which is optionally substituted with 1, 2, 3, or 4 independently selected R^{e} groups;
G^{3E}, at each occurrence, is independently C₃-C₈ monocyclic cycloalkyl or 4-11 membered heterocycle; wherein each G^{3E} is optionally substituted with 1, 2, 3, or 4 substituents independently selected from the group consisting of R^{e} and G^{3F};
G^{3F}, at each occurrence, is independently a 4-7 membered monocyclic heterocycle or a C₃-C₆ monocyclic cycloalkyl; wherein each G^{3F} is optionally substituted with 1, 2, 3, or 4 independently selected R^{e} groups;
R^{e}, at each occurrence, is independently C₂-C₆ alkenyl, C₂-C₆ alkynyl, C₁-C₆ alkyl, C₁-C₆ haloalkyl, halogen, oxo, —CN, —N₃, NO₂, —OR^{f}, —OC(O)R^{g}, —OC(O)NR^{f}R^{h}, —SR^{f}, —S(O)₂R^{f}, —S(O)₂NR^{f}R^{h}, —C(O)R^{f}, —C(O)OR^{f}, —C(O)NR^{f}R^{h}, —C(O)N(R^{h})S(O)₂R^{f}, —N(R^{f})₂, —N(R^{h})C(O)R^{f}, —N(R^{h})S(O)₂R^{g}, —N(R^{h})C(O)O(R^{g}), —N(R^{h})C(O)N R^{f}R^{h}, or —N(R^{h})S(O)₂NR^{f}R^{h}; wherein the C₁-C₆ haloalkyl and the C₁-C₆ alkyl are each optionally substituted with 1 or 2 substituents independently selected from the group consisting of halogen, —CN, NO₂, —OR^{f}, —OC(O)R^{g}, —OC(O)NR^{f}R^{h}, —SR^{f}, —S(O)₂R^{f}, —S(O)₂NRfR^{h}, —C(O)R^{f}, —C(O)OR^{f}, —C(O)NR^{f}R^{h}, —C(O)N(R^{h})S(O)₂R^{f}, —N(R^{f})₂, —N(R^{h})C(O)R, —N(R^{h})S(O)₂R^{g}, —N(R^{h})C(O)O(R^{g}), —N(R^{h})C(O)N R^{f}R^{h}, and —N(R^{h})S(O)₂NR^{f}R^{h};
R^{f}, at each occurrence, is independently hydrogen, C₁-C₆ alkyl, C₂-C₆ alkenyl, C₂-C₆ alkynyl, C₁-C₆ haloalkyl, —(C₁-C₆ alkylenyl)-CN, —(C₁-C₆ alkylenyl)-OR^{m}, —(C₁-C₆ alkylenyl)-OC(O)Rⁿ, —(C₁-C₆ alkylenyl)-OC(O)N(R^{m})₂, —(C₁-C₆ alkylenyl)-SR^{m}, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-C(O)N(R^{m})S(O)₂Rⁿ, —(C₁-C₆ alkylenyl)-N(R^{m})₂, —(C₁-C₆ alkylenyl)-N(R^{m})C(O)Rⁿ, —(C₁-C₆ alkylenyl)-N(R^{m})S(O)₂Rⁿ, —(C₁-C₆ alkylenyl)-N(R^{m})C(O)O(Rⁿ), —(C₁-C₆ alkylenyl)-N(R^{m})C(O)N(R^{m})₂, or —(C₁-C₆ alkylenyl)-N(R^{m})S(O)₂N(R^{m})₂;
R^{g}, at each occurrence, is independently C₁-C₆ alkyl, C₂-C₆ alkenyl, C₂-C₆ alkynyl, C₁-C₆ haloalkyl, —(C₁-C₆ alkylenyl)-CN, —(C₁-C₆ alkylenyl)-OR^{m}, —(C₁-C₆ alkylenyl)-OC(O)Rⁿ, —(C₁-C₆ alkylenyl)-OC(O)N(R^{m})₂, —(C₁-C₆ alkylenyl)-SR^{m}, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-C(O)N(R^{m})S(O)₂Rⁿ, —(C₁-C₆ alkylenyl)-N(R^{m})₂, —(C₁-C₆ alkylenyl)-N(R^{m})C(O)Rⁿ, —(C₁-C₆ alkylenyl)-N(R^{m})S(O)₂Rⁿ, —(C₁-C₆ alkylenyl)-N(R^{m})C(O)O(Rⁿ), —(C₁-C₆ alkylenyl)-N(R^{m})C(O)N(R^{m})₂, or —(C₁-C₆ alkylenyl)-N(R^{m})S(O)₂N(R^{m})₂;
R^{h}, at each occurrence, is independently hydrogen, C₁-C₆ alkyl, C₁-C₆ haloalkyl, or —(C₁-C₆ alkylenyl)-OR^{m};
R^{1a}, R^{1b}, R^{1c}, R^{1d}, and R^{2a}, at each occurrence, are each independently C₁-C₆ alkyl, C₂-C₆ alkenyl, C₂-C₆ alkynyl, halogen, C₁-C₆ haloalkyl, oxo, —CN,
NO₂, —OR^{m}, —OC(O)Rⁿ, —OC(O)N(R^{m})₂, —SR^{m}, —S(O)₂R^{m}, —S(O)₂N(R^{m})₂, —C(O)R^{m}, —C(O)OR^{m}, —C(O)N(R^{m})₂, —C(O)N(R^{m})S(O)₂Rⁿ, —N(R^{m})₂, —N(R^{m})(alkoxyalkyl), —N(alkoxyalkyl)₂, —N(R^{m})C(O)Rⁿ, —N(R^{m})S(O)₂Rⁿ, —N(R^{m})C(O)O(Rⁿ), —N(R^{m})C(O)N(R^{m})₂, —N(R^{m})S(O)₂N(R^{m})₂, —(C₁-C₆ alkylenyl)-CN, —(C₁-C₆ alkylenyl)-OR^{m}, —(C₁-C₆ alkylenyl)-OC(O)Rⁿ, —(C₁-C₆ alkylenyl)-OC(O)N(R^{m})₂, —(C₁-C₆ alkylenyl)-SR^{m}, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-C(O)N(R^{m})S(O)₂Rⁿ, —(C₁-C₆ alkylenyl)-N(R^{m})₂, —(C₁-C₆ alkylenyl)-N(R^{m})C(O)Rⁿ, —(C₁-C₆ alkylenyl)-N(R^{m})S(O)₂Rⁿ, —(C₁-C₆ alkylenyl)-N(R^{m})C(O)O(Rⁿ), —(C₁-C₆ alkylenyl)-N(R^{m})C(O)N(Rⁿ)₂, or —(C₁-C₆ alkylenyl)-N(R^{m})S(O)₂N(Rⁿ)₂;
R^{m}, at each occurrence, is independently hydrogen, C₁-C₆ alkyl, or C₁-C₆ haloalkyl;
Rⁿ, at each occurrence, is independently C₁-C₆ alkyl or C₁-C₆ haloalkyl; and
R⁴ is hydrogen, C₁-C₃ alkyl, or C₁-C₃ haloalkyl;
with the proviso that when R¹ is C₁-C₆ alkyl or G^{1A}, wherein G^{1A} is optionally substituted phenyl, optionally substituted 5-6 membered monocyclic heteroaryl, or optionally substituted 4-7 membered monocyclic heterocycle, R² is C₁-C₆ alkyl, and R³ is G^{3A}, then G^{3A} is not optionally substituted phenyl or optionally substituted 5-6 membered monocyclic heteroaryl.

As used herein, the term “therapeutic combination” or “combination” means a combination of P with one or two correctors C1 and/or C2.

The correctors C1 and C2 when used together provide a synergetic/additive effect on the expression level and/or function of mutant CFTR.

While not limited to any particular mode of action, C1 and C2 correctors may act via different mechanisms. More specifically, C1 and C2 correctors bind to CFTR protein in the cells. Such binding can be measured using the Patch Clamp assay (TECC) and Molecular Sensing technology as described herein.

In a particular embodiment of the combination of P potentiator with two correctors, C2 corrector does not act through MSD1 domain of CFTR, and C1 corrector acts through MSD1 domain of CFTR. More particular C1 corrector and the C2 corrector bind to different portions of the CFTR protein. Specifically C1 and C2 correctors bind to different domains of CFTR protein. In some embodiments C1 corrector is a corrector that binds to MSD1 domain of CFTR protein. In some embodiments C2 corrector is a corrector that does not bind to MSD1 domain of CFTR protein.

In a particular embodiment the binding constant (K_{d}) of the C2 corrector to membrane fractions of CFTR expressing cells is more than 200, 300, 400, 500, 600 nM as measured using molecular sensing technology. In a particular embodiment the binding constant (K_{d}) of the C1 corrector to membrane fractions of CFTR expressing cells is less than 50, 100, 200, 300 nM as measured using molecular sensing technology.

In a particular embodiment the combination of P with C1 and C2 provides an effect on the short circuit (I_{sc}) current as measured by the trans epithelial clamp circuit assay (TECC assay) as disclosed herein, that is at least equal to 85% of the sum of the individual I_{sc} of the C1 corrector and C2 corrector in the same cells. In particular embodiment I_{sc} is at least 90% of the sum of the individual I_{sc} of the C1 corrector and C2 in the same cells.

More specifically the short circuit (I_{sc}) current as measured by the TECC assay on F508del homozygous patient derived cells using the combination of P with C1 and C2 yields at least 30, 35, 40, 45, 50, 60, 75, 80, 85, or 90% of the I_{sc} obtainable with the CFTR protein according to SEQ ID NO: 1 as measured by said TECC assay.

In a particular embodiment said combination of P potentiator with C1 or C2 corrector said combination produces an additional transepithelial conductance (ΔGt) of at least 2, at least 1.5, at least 1, at least 0.5, at least 0.25 mS/cm2 as measured using transepithelial clap circuit assay in the W1282X Fisher rat thyroid (FRT) cells. More particular said combination of P potentiator with C1 or C2 corrector said combination produces an additional transepithelial conductance (ΔGt) of at least 1 mS/cm² as measured using transepithelial clap circuit assay in the W1282X Fisher rat thyroid (FRT) cells. In another embodiment said combination of P potentiator with C1 corrector and C2 corrector said combination produces an additional transepithelial conductance (ΔGt) of at least 3.5, at least 3, at least 2, at least 1.5, at least 1 mS/cm² as measured using transepithelial clap circuit assay the W1282X Fisher rat thyroid (FRT) cells.

P potentiator, C1 corrector and C2 corrector may be used in the form of pharmaceutically acceptable salts. Pharmaceutically acceptable salts have been described in S. M. Berge et al. J. Pharmaceutical Sciences, 1977, 66: 1-19.

P, C1 and C2 may contain either a basic or an acidic functionality, or both, and can be converted to a pharmaceutically acceptable salt, when desired, by using a suitable acid or base. The salts may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of P, C1 and C2 by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds P, C1 and C2 described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### Uses of the Combinations

In one aspect the present invention provides a method of treatment of cystic fibrosis in a subject comprising the steps of:
a) analyzing the sequence of cystic fibrosis transmembrane conductance regulator (CFTR) protein from the subject for the presence of a premature termination codon (PTC) or a nonsense mutation,
b) identifying a subject having a mutation located between the amino acid residues 1164-1480 of SEQ ID NO: 1, and
c) administering a combination comprising:
   i. a modulator of the function (P potentiator) of cystic fibrosis transmembrane conductance regulator (CFTR) protein,
   ii. a modulator of the cellular processing and/or localization (C corrector), wherein said C corrector is not a read-through corrector,
      wherein said combination does not comprise a read-through agent, and wherein said combination produces an additional transepithelial conductance (ΔGt) of at least 1 mS/cm2 as measured using transepithelial clap circuit assay in the W1282X Fisher rat thyroid (FRT) cells.

In another aspect the present invention provides a method of treatment of cystic fibrosis in a subject comprising the steps of:
a) analyzing the sequence of cystic fibrosis transmembrane conductance regulator (CFTR) protein from the subject for the presence of a premature termination codon (PTC) or a nonsense mutation,
b) identifying a subject having a mutation located between the amino acid residues 1164-1480 of SEQ ID NO: 1, and
c) administering a combination comprising:
   i. a modulator of the function (P potentiator) of cystic fibrosis transmembrane conductance regulator (CFTR) protein,
   ii. a modulator of the cellular processing and/or localization (C corrector), wherein said C corrector is not a read-through corrector, and
   iii. a second modulator of the cellular processing and/or localization (a second C corrector), wherein said second C corrector is not a read-through corrector,
      wherein said combination does not comprise a read-through agent, and wherein said combination produces an additional transepithelial conductance (ΔGt) of at least 3.5 mS/cm2 as measured using transepithelial clap circuit assay in the W1282X Fisher rat thyroid (FRT) cells.

The present invention further provides a pharmaceutical combination comprising
i. a modulator of the function (P potentiator) of cystic fibrosis transmembrane conductance regulator (CFTR) protein,
ii. a modulator of the cellular processing and/or localization (C corrector), wherein said C corrector is not a read-through corrector,
   for use in the treatment of cystic fibrosis in a subject having a mutation located between the amino acid residues 1164-1480 of SEQ ID NO: 1,
   wherein said combination does not comprise a read-through agent, and wherein said combination produces an additional transepithelial conductance (ΔGt) of at least 1 mS/cm2 as measured using transepithelial clap circuit assay in the W1282X Fisher rat thyroid (FRT) cells.

The present invention also provides a pharmaceutical combination comprising
i. a modulator of the function (P potentiator) of cystic fibrosis transmembrane conductance regulator (CFTR) protein,
ii. a modulator of the cellular processing and/or localization (C corrector), wherein said C corrector is not a read-through corrector and
iii. a second modulator of the cellular processing and/or localization (second C corrector), wherein said second C corrector is not a read-through corrector
   for use in the treatment of cystic fibrosis in a subject having a mutation located between the amino acid residues 1164-1480 of SEQ ID NO: 1, wherein said combination does not comprise a read-through agent, and wherein said combination produces an additional transepithelial conductance (ΔGt) of at least 3.5 mS/cm2 as measured using transepithelial clap circuit assay in the W1282X Fisher rat thyroid (FRT) cells.

In yet another embodiment the present invention provides use of a combination comprising:
i. a modulator of the function (P potentiator) of cystic fibrosis transmembrane conductance regulator (CFTR) protein,
ii. a modulator of the cellular processing and/or localization (C corrector), wherein said C corrector is not a read-through corrector
   or pharmaceutically acceptable salts thereof in the preparation of a medicament for the treatment of cystic fibrosis in a subject having a mutation located between the amino acid residues 1164-1480 of SEQ ID NO: 1,
   wherein said combination does not comprise a read-through agent, and wherein said combination produces an additional transepithelial conductance (ΔGt) of at least 1 mS/cm2 as measured using transepithelial clap circuit assay in the W1282X Fisher rat thyroid (FRT) cells.

In yet another embodiment the present invention provides use of a combination comprising:
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, and
iii. a second modulator of the cellular processing and/or localization (second C corrector), wherein said second C corrector is not a read-through corrector
   or pharmaceutically acceptable salts thereof in the preparation of a medicament for the treatment of cystic fibrosis in a subject having a mutation located between the amino acid residues 1164-1480 of SEQ ID NO: 1,
   wherein said combination does not comprise a read-through agent, and wherein said combination produces an additional transepithelial conductance (ΔGt) of at least 3.5 mS/cm2 as measured using transepithelial clap circuit assay in the W1282X Fisher rat thyroid (FRT) cells.

The following variations of the methods, compositions and uses are provided.

In a specific embodiment the cystic fibrosis results from a Class I mutation in CFTR protein, wherein said CFTR protein comprises a premature termination codon (PTC) or a nonsense mutation, and wherein said mutation is located between the amino acid residues 1164-1480 of SEQ ID NO: 1

In a particular embodiment the premature termination codon (PTC) or a nonsense mutation is UGA codon (or opal codon).

In a more specific embodiment said mutation is W1282X mutation.

In one embodiment C corrector is not acting through the membrane spanning domain 1 (MSD1) domain of CFTR. In yet another embodiment C corrector is acting through the membrane spanning domain 1 (MSD1) domain of CFTR. In a particular embodiment of the combination of P potentiator with C corrector, said C corrector binds to MSD1 domain of CFTR protein. In yet another embodiment said C corrector does not bind to MSD1 domain of CFTR protein.

In a particular embodiment C corrector is either C1 corrector or C2 corrector.

In a particular embodiment of the combination of P potentiator with C corrector and second C corrector, said correctors act via different mechanisms. More specifically, said correctors bind to CFTR protein. In a more particular embodiment said correctors bind to different domains of CFTR protein. In a more specific embodiment one of the correctors binds to MSD1 domain of CFTR protein, while the second corrector does not bind to MSD1 domain of CFTR protein.

In a particular embodiment of the combination of P potentiator with C corrector and second C corrector, said C corrector is a C1 corrector and said second C corrector is a C2 corrector, wherein said correctors bind to different portions of the CFTR protein. In a more particular embodiment C1 and C2 correctors bind to different domains of CFTR protein. In a particular embodiment C1 corrector is a corrector that binds to MSD1 domain of CFTR protein. In some embodiments C2 corrector is a corrector that does not bind to MSD1 domain of CFTR protein.

In a particular embodiment said combination of P potentiator with C1 or C2 corrector said combination produces an additional transepithelial conductance (ΔGt) of at least 2, at least 1.5, at least 1, at least 0.5, at least 0.25 mS/cm2 as measured using transepithelial clap circuit assay in the W1282X Fisher rat thyroid (FRT) cells. More particular said combination of P potentiator with C1 or C2 corrector said combination produces an additional transepithelial conductance (ΔGt) of at least 1 mS/cm2 as measured using transepithelial clap circuit assay in the W1282X Fisher rat thyroid (FRT) cells. In another embodiment said combination of P potentiator with C1 corrector and C2 corrector said combination produces an additional transepithelial conductance (ΔGt) of at least 3.5, at least 3, at least 2, at least 1.5, at least 1 mS/cm2 as measured using transepithelial clap circuit assay the W1282X Fisher rat thyroid (FRT) cells.

The present invention also provides a method of treatment of cystic fibrosis in a subject comprising the steps of:
a) analyzing the sequence of cystic fibrosis transmembrane conductance regulator (CFTR) protein from the subject for the presence of a premature termination codon (PTC) or a nonsense mutation,
b) identifying a subject having a mutation located between the amino acid residues 1164-1480 of SEQ ID NO: 1, and
c) administering a combination comprising:
   i. a modulator of the function (P potentiator) of cystic fibrosis transmembrane conductance regulator (CFTR) protein,
   ii. a modulator of the cellular processing and/or localization (C corrector), wherein said C corrector is not a read-through corrector, and wherein said C corrector is not acting through the membrane spanning domain 1 (MSD1) domain of CFTR,
      wherein said combination does not comprise a read-through agent.

In another embodiment the present invention provides a method of treatment of cystic fibrosis in a subject comprising the steps of:
a) analyzing the sequence of cystic fibrosis transmembrane conductance regulator (CFTR) protein from the subject for the presence of a premature termination codon (PTC) or a nonsense mutation,
b) identifying a subject having amutation located between the amino acid residues 1164-1480 of SEQ ID NO: 1, and
c) administering a combination comprising:
   i. a modulator of the function (P potentiator) of cystic fibrosis transmembrane conductance regulator (CFTR) protein,
   ii. a modulator of the cellular processing and/or localization (C corrector), wherein said C corrector is not a read-through corrector, and wherein said C corrector is acting through the membrane spanning domain 1 (MSD1) domain of CFTR,
      wherein said combination does not comprise a read-through agent.

In another aspect the present invention provides a method of treatment of cystic fibrosis in a subject comprising the steps of:
a) analyzing the sequence of cystic fibrosis transmembrane conductance regulator (CFTR) protein from the subject for the presence of a premature termination codon (PTC) or a nonsense mutation,
b) identifying a subject having a mutation located between the amino acid residues 1164-1480 of SEQ ID NO: 1, and
c) administering a combination comprising:
   i. a modulator of the function (P potentiator) of cystic fibrosis transmembrane conductance regulator (CFTR) protein,
   ii. a modulator of the cellular processing and/or localization (C corrector), wherein said C corrector is not a read-through corrector, and wherein said C corrector is not acting through the membrane spanning domain 1 (MSD1) domain of CFTR and
   iii. a second modulator of the cellular processing and/or localization (a second C corrector), wherein said C corrector is not a read-through corrector, and wherein said C corrector is acting through the membrane spanning domain 1 (MSD1) domain of CFTR,
      wherein said combination does not comprise a read-through agent.

The present invention further provides a pharmaceutical combination comprising
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, and wherein said C corrector is not acting through the membrane spanning domain 1 (MSD1) domain of CFTR for use in the treatment of cystic fibrosis in a subject having a mutation located between the amino acid residues 1164-1480 of SEQ ID NO: 1,
   wherein said combination does not comprise a read-through agent.

The present invention further provides a pharmaceutical combination comprising
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, and wherein said C corrector is acting through the membrane spanning domain 1 (MSD1) domain of CFTR for use in the treatment of cystic fibrosis in a subject having a mutation located between the amino acid residues 1164-1480 of SEQ ID NO: 1,
   wherein said combination does not comprise a read-through agent.

The present invention also provides a pharmaceutical combination comprising
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, and wherein said C corrector is not acting through the membrane spanning domain 1 (MSD1) domain of CFTR and
iii. a second modulator of the cellular processing and/or localization (second C corrector), wherein said C corrector is not a read-through corrector, and wherein said C corrector is acting through the membrane spanning domain 1 (MSD1) domain of CFTR.
   for use in the treatment of cystic fibrosis in a subject having a mutation located between the amino acid residues 1164-1480 of SEQ ID NO: 1, wherein said combination does not comprise a read-through agent.

In yet another embodiment the present invention provides use of a combination comprising:
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, and wherein said C corrector is not acting through the membrane spanning domain 1 (MSD1) domain of CFTR.
   or pharmaceutically acceptable salts thereof in the preparation of a medicament for the treatment of cystic fibrosis in a subject having a mutation located between the amino acid residues 1164-1480 of SEQ ID NO: 1,
   wherein said combination does not comprise a read-through agent.

In yet another embodiment the present invention provides use of a combination comprising:
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, and wherein said C corrector is acting through the membrane spanning domain 1 (MSD1) domain of CFTR.
   or pharmaceutically acceptable salts thereof in the preparation of a medicament for the treatment of cystic fibrosis in a subject having a mutation located between the amino acid residues 1164-1480 of SEQ ID NO: 1,
   wherein said combination does not comprise a read-through agent.

In yet another embodiment the present invention provides use of a combination comprising:
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, and wherein said C corrector is not acting through the membrane spanning domain 1 (MSD1) domain of CFTR. and
iii. a second modulator of the cellular processing and/or localization (second C corrector), wherein said C corrector is not a read-through corrector, and wherein said C corrector is acting through the membrane spanning domain 1 (MSD1) domain of CFTR
   or pharmaceutically acceptable salts thereof in the preparation of a medicament for the treatment of cystic fibrosis in a subject having a mutation located between the amino acid residues 1164-1480 of SEQ ID NO: 1,
   wherein said combination does not comprise a read-through agent.

The following variations of the above methods, compositions and uses are provided.

In a specific embodiment the cystic fibrosis results from a Class I mutation in CFTR protein, wherein said CFTR protein comprises a premature termination codon (PTC) or a nonsense mutation, and wherein said mutation is located between the amino acid residues 1164-1480 of SEQ ID NO: 1

In a particular embodiment the premature termination codon (PTC) or a nonsense mutation is UGA codon (or opal codon).

In a more specific embodiment said mutation is W1282X mutation.

In a particular embodiment of the combination of P potentiator with C corrector that is acting through MSD1 domain of CFTR, said C corrector binds to MSD1 domain of CFTR protein. In yet another embodiment said C corrector that is not acting through MSD1 domain of CFTR does not bind to MSD1 domain of CFTR protein.

In a particular embodiment C corrector that is acting through MSD1 domain of CFTR is C1 corrector as described herein. In another embodiment C corrector that is not acting through MSD1 domain of CFTR is C2 corrector as described herein.

In another embodiment of the combination of P potentiator with C corrector and second C corrector, said correctors act via different mechanisms. In particular aspect, said correctors bind to CFTR protein. In a more particular embodiment said correctors bind to different domains of CFTR protein. In a more specific embodiment of the combination of P potentiator with two correctors one of the correctors not acting through MSD1 domain of CFTR does not bind to MSD1 domain of CFTR protein, while the second C corrector acting through MSD1 domain of CFTR binds to MSD1 domain of CFTR protein.

In a particular embodiment of the combination of P potentiator with C corrector and second C corrector, said C corrector is a C2 corrector and said second C corrector is a C1 corrector, wherein said C2 corrector does not acts through MSD1 domain of CFTR, and said C1 corrector acts through MSD1 domain of CFTR. In a particular embodiment of the combination of P potentiator with C corrector and second C corrector, said C corrector is a C2 corrector and said second C corrector is a C1 corrector, wherein said correctors bind to different portions of the CFTR protein. In a more particular embodiment C1 and C2 correctors bind to different domains of CFTR protein. In a particular embodiment C1 corrector is a corrector that binds to MSD1 domain of CFTR protein. In some embodiments C2 corrector is a corrector that does not bind to MSD1 domain of CFTR protein.

In a particular embodiment said combination of P potentiator with C1 or C2 corrector said combination produces an additional transepithelial conductance (ΔGt) of at least 2, at least 1.5, at least 1, at least 0.5, at least 0.25 mS/cm2 as measured using transepithelial clap circuit assay (TECC) in the W1282X Fisher rat thyroid (FRT) cells. More particular said combination of P potentiator with C1 or C2 corrector said combination produces an additional transepithelial conductance (ΔGt) of at least 1 mS/cm2 as measured using transepithelial clap circuit assay in the W1282X Fisher rat thyroid (FRT) cells. In another embodiment said combination of P potentiator with C1 corrector and C2 corrector said combination produces an additional transepithelial conductance (ΔGt) of at least 3.5, at least 3, at least 2, at least 1.5, at least 1 mS/cm2 as measured using transepithelial clap circuit assay the W1282X Fisher rat thyroid (FRT) cells.

In one embodiment P potentiator is a compound according to formula (I) or formula (II), or a pharmaceutically acceptable salt thereof. In one embodiment C corrector is a compound according to formula (III), or a pharmaceutically acceptable salt thereof, or, alternatively, a compound according to formula (IV) or formula (V), or a pharmaceutically acceptable salt thereof.

In one embodiment C1 corrector is a compound according to formula (III), or a pharmaceutically acceptable salt thereof, and C2 corrector is a compound according to formula (IV) or formula (V), or a pharmaceutically acceptable salt thereof.

In a particular embodiment the P potentiator is selected from

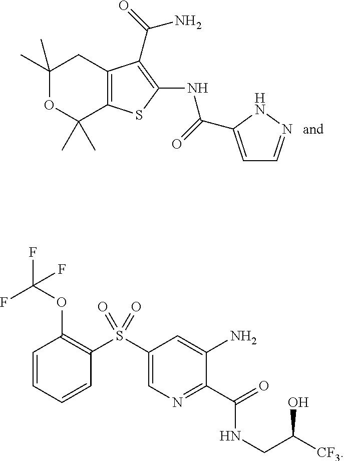 [SMILES: CC1(C)Cc2c(sc(NC(=O)c3ccn[nH]3)c2C(N)=O)C(C)(C)O1 ; Nc1cc(S(=O)(=O)c2ccccc2OC(F)(F)F)cnc1C(=O)NC[C@@H](O)C(F)(F)F]

In a particular embodiment the C1 corrector is:

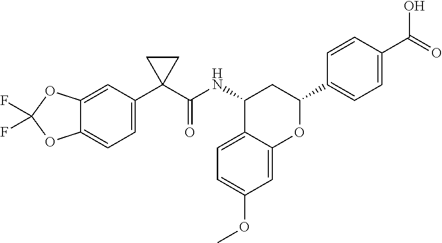 [SMILES: COc1ccc2c(c1)O[C@@H](c1ccc(C(=O)O)cc1)C[C@H]2NC(=O)C1(c2ccc3c(c2)OC(F)(F)O3)CC1]

In a particular embodiment the C2 corrector is selected from the compounds according to formula (IV) or (V), or a pharmaceutically acceptable salt thereof and the C1 corrector is

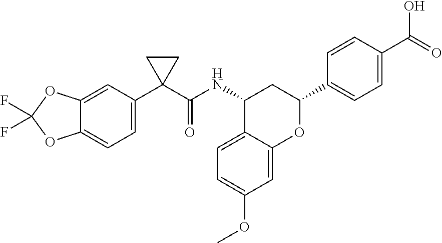 [SMILES: COc1ccc2c(c1)O[C@@H](c1ccc(C(=O)O)cc1)C[C@H]2NC(=O)C1(c2ccc3c(c2)OC(F)(F)O3)CC1]

### Pharmaceutical Compositions and Formulations

P, C1 and C2 compounds are typically administered in the form of a pharmaceutical composition. Such compositions can be prepared in a manner well known in the pharmaceutical art and comprise a therapeutically effective amount of a compound P, C1 and C2, or a pharmaceutically acceptable salt thereof together with a pharmaceutically acceptable carrier. The phrase “pharmaceutical composition” refers to a composition suitable for administration in medical or veterinary use.

The pharmaceutical compositions that comprise P, C1 and C2, alone or in combination with further therapeutically active ingredient(s), may be administered to the subjects orally, rectally, parenterally, intracistemally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which may serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous diluents, solvents, or vehicles include water, ethanol, polyols (such as glycerol, propylene glycol, polyethylene glycol and the like), vegetable oils (such as olive oil), injectable organic esters (such as ethyl oleate), and suitable mixtures thereof. Proper fluidity may be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

In some cases, in order to prolong the effect of the drug, it may be desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations may be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In certain embodiments, solid dosage forms may contain from 1% to 95% (w/w) of a compound P, C1 and C2. In certain embodiments, the compounds P, C1 and C2, or pharmaceutically acceptable salts thereof, may be present in the solid dosage form in a range of from 5% to 70% (w/w). In such solid dosage forms, the active compound may be mixed with at least one inert, pharmaceutically acceptable carrier, such as sodium citrate or dicalcium phosphate and/or a), fillers or extenders such as starches, lactose, sucrose, glucose, mannitol, and silicic acid; b) binders such as carboxymethylcellulose, alginates, gelatin, polyvinylpyrrolidone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

The pharmaceutical composition may be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form may be a capsule, tablet, cachet, or lozenge itself, or it may be the appropriate number of any of these in packaged form. The quantity of active component in a unit dose preparation may be varied or adjusted from 0.1 mg to 1000 mg, from 1 mg to 100 mg, or from 1% to 95% (w/w) of a unit dose, according to the particular application and the potency of the active component. The composition may, if desired, also contain other compatible therapeutic agents.

### Administration

The dose to be administered to a subject may be determined by the efficacy of the particular P, C1 and C2 compound(s) employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician may evaluate factors such as the circulating plasma levels of the compound, compound toxicities, and/or the progression of the disease, etc.

For administration, compounds P, C1 and C2 may be administered at a rate determined by factors that may include, but are not limited to, the LD₅₀ of the compound, the pharmacokinetic profile of the compound, contraindicated drugs, and the side-effects of the compound at various concentrations, as applied to the mass and overall health of the subject. Administration may be accomplished via single or divided doses.

The compounds P, C1 and C2 utilized in the pharmaceutical method of the invention may be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Treatment may be initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds may also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols, and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which may be prepared by mixing the compounds with suitable non-irritating carriers or carriers such as cocoa butter, polyethylene glycol, or a suppository wax which are solid at room temperature but liquid at body temperature and therefore melt in the rectum or vaginal cavity and release the active compound.

Compounds may also be administered in the form of liposomes. Liposomes generally may be derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes may be used. The present compositions in liposome form may contain, in addition to a compound of the invention, stabilizers, preservatives, excipients, and the like. Examples of lipids include, but are not limited to, natural and synthetic phospholipids, and phosphatidyl cholines (lecithins), used separately or together.

Methods to form liposomes have been described, see example, Prescott, Ed., Methods in Cell Biology, Volume XIV, Academic Press, New York, N.Y. (1976), p. 33 et seq.

Dosage forms for topical administration of a compound described herein include powders, sprays, ointments, and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

A compounds P, C1 and C2 may also be administered in sustained release forms or from sustained release drug delivery systems.

### Co-Administration

As used herein, the term “therapeutic combination” or “combination” means a combination of P with one or two correctors that are either (i) administered to a patient in need thereof simultaneously in separate formulations or in a single formulation; or (ii) administered to a patient in need thereof at different time points as part of a treatment regimen.

In a particular embodiment P potentiator is administered subsequently after the administration of C1 and/or C2 corrector. In yet another embodiment C1 and/or C2 and P are administered simultaneously.

The compounds P, C1 and C2 may be administered in its combination or they may be co-administered with other therapeutic agents, including other compounds that demonstrate the same or a similar therapeutic activity and that are determined to be safe and efficacious for such combined administration. The term “co-administered” means the administration of two or more different therapeutic agents to a subject in a single pharmaceutical composition or in separate pharmaceutical compositions. Thus co-administration involves administration at the same time of a single pharmaceutical composition comprising two or more therapeutic agents or administration of two or more different compositions to the same subject at the same or different times.

The compounds P, C1 and C2 may be co-administered with a therapeutically effective amount of one or more therapeutic agents to treat a CFTR mediated disease, where examples of the agents include, but are not limited to antibiotics (for example, aminoglycosides, colistin, aztreonam, ciprofloxacin, and azithromycin), expectorants (for example, hypertonic saline, acetylcysteine, dornase alfa, and denufosol), pancreatic enzyme supplements (for example, pancreatin, and pancrelipase), CFTR potentiators, and CFTR correctors. In one embodiment, the CFTR mediated disease is cystic fibrosis. In one embodiment, the compounds P, C1 and C2 or pharmaceutically acceptable salts thereof may be co-administered with an additional potentiator and one or more additional correctors.

### Subjects Suitable for Treatment

Subjects suitable for treatment with the methods and combinations of the present invention include individuals having mutant-CFTR protein-mediated condition disorder or disease, or symptom of such condition, disorder, or disease that results from or is correlated to the presence of a mutant-CFTR. In particular said subjects have two alleles of the mutant CFTR. More specifically the subjects suitable for the treatment are having a mutation in the CFTR protein between amino acid residues 1164-1480 of the wild type CFTR. More particular said mutation is a premature termination codon (PTC) or a nonsense mutation. Subjects suitable for treatment using the methods or the combinations of the present invention include any organism carrying said mutations. In particular said subjects suitable for treatment are humans with CF having a mutation in the CFTR protein between amino acid residues 1164-1480 of the wild type CFTR. More particular said mutation is a PTC or a nonsense mutation.

Symptoms of mutant-CFTR protein-mediated conditions are well known to a skilled person and include meconium ileus, liver disease including biliary tract obstruction and stenosis, pancreatic insufficiency, pulmonary disease including chronic bacterial infections and other infections of the lung.

The combinations of the present invention affect the processing and the chloride ion transport capability of the mutant-CFTR by increasing the reduced level of ion transport mediated by a mutant-CFTR having a mutation located between the amino acid residues 1164-1480 of the wild type CFTR. The combinations of the present invention are useful in treating patients that have Class I defects in the CFTR gene, which result in a mutant-CFTR or low levels of CFTR or a CFTR that has reduced chloride conductance capability due to folding or cellular processing defects. In particular the combinations of the present invention are useful in the treatment of mutations in the CFTR protein between amino acid residues 1164-1480 of the wild type CFTR. More specifically said mutation is a PTC or a nonsense mutation. More particular the methods and the combinations of the present invention are useful in the treatment of subjects having a W1284X mutation in the CFTR protein.

A subject suitable for treatment with a method of the present invention may be homozygous for a specific mutant-CFTR. More specifically homozygous subjects have two copies of a specific mutant-CFTR having a mutation between amino acid residues 1164-1480 of the wild type CFTR. In addition, subjects suitable for treatment with the methods and the combinations of the present invention may also be heterozygous for two different CFTR mutants, i.e., wherein the genome of the subjects includes two different mutant forms of CFTR, wherein at least one of said forms is a mutant-CFTR having a mutation between amino acid residues 1164-1480 of the wild type CFTR. More specifically said mutation is a PTC or a nonsense mutation.

### Methods of Detecting CFTR Mutations

The process of analysis of the sequence of CFTR protein from the subject for the presence of a premature termination codon (PTC) or a nonsense mutation includes any suitable method, of which many are known to a skilled person. Suitable methods include determining the DNA sequence, or by detecting an RNA transcript corresponding to such DNA sequence, of a polymorphic gene. Various other detection techniques suitable for use in the methods will be apparent to a skilled person familiar with methods of detecting, identifying, and/or distinguishing CFTR mutations. Such detection techniques include but are not limited to direct sequencing, use of “molecular beacons” as described in Marras et al., 1999, electrochemical detection as described in U.S. Pat. No. 5,871,918, rolling circle amplification as described in Gusev et al, 2001, and a non-PCR based detection method as described in Lieder, Advance for Laboratory Managers, 70 (2000).

Methods for detecting CFTR gene mutations have been also described in e.g., Audrezet et al, “Genomic rearrangements in the CFTR gene: extensive allelic heterogeneity and diverse mutational mechanisms” Hum Mutat. 2004 April; 23(4):343-57, WO2004/040013 and U.S. Pat. No. 7,741,028 herein incorporated by reference.

Suitable biological specimens useful for analyzing for the presence of a CFTR mutation in the subject are those which comprise cells and DNA and include, but are not limited to blood or blood components, dried blood spots, urine, buccal swabs and saliva.

### Kits

In another embodiment the present invention provides a kit comprising:
i. a pharmaceutical composition comprising a P potentiator;
ii. a pharmaceutical composition comprising a C corrector, wherein said C corrector is not a read-through corrector;
iii. instructions for using said kit for treating cystic fibrosis in a subject having a mutation located between the amino acid residues 1164-1480 of SEQ ID NO: 1,
   wherein said kit does not comprise a read-through agent, and
   wherein said combination produces an additional transepithelial conductance (ΔGt) of at least 1 mS/cm2 as measured using transepithelial clap circuit assay (TECC assay) in the W1282X Fisher rat thyroid (FRT) cells.

In another embodiment the present invention provides a kit comprising:
i. a pharmaceutical composition comprising a P potentiator;
ii. a pharmaceutical composition comprising a C corrector, wherein said C corrector is not a read-through corrector, wherein said corrector is not acting through the membrane spanning domain 1 (MSD1) of CFTR;
iii. instructions for using said kit for treating cystic fibrosis in a subject having a mutation located between the amino acid residues 1164-1480 of SEQ ID NO: 1,
   wherein said kit does not comprise a read-through agent.

In another embodiment the present invention provides a kit comprising:
i. a pharmaceutical composition comprising a P potentiator;
ii. a pharmaceutical composition comprising a C corrector, wherein said C corrector is not a read-through corrector, wherein said corrector is acting through the membrane spanning domain 1 (MSD1) of CFTR;
iii. instructions for using said kit for treating cystic fibrosis in a subject having a mutation located between the amino acid residues 1164-1480 of SEQ ID NO: 1,
   wherein said kit does not comprise a read-through agent.

In a particular embodiment said kits further comprise a pharmaceutical composition comprising a second C corrector, wherein said corrector is not a read-through corrector.

In a particular embodiment said kits comprise further additional components. Such optional components of the kit may include buffers, delivery vehicles, delivery means, etc. for administering of the potentiator and one or two corrector compounds, and/or for performing a diagnostic assay. The various components of the kit may be present in separate containers or certain components may be combined into a single container. The kits also may include one or more additional pharmaceuticals or agents for treating a subject having a mutant-CFTR protein. Yet in another embodiment, the kit may further include a system for characterizing mutant-CFTR.

In certain embodiments the kit may include a single pharmaceutical composition comprising a combination of the potentiator with one or two correctors present as one or more unit dosages. In yet other embodiments, the kits may include two or more separate pharmaceutical compositions comprising said potentiator with one or two correctors or combinations thereof.

In a particular embodiment C corrector is a C1 corrector as described herein. In yet another embodiment said C corrector is a C2 corrector as described herein. In another embodiment said C corrector is a C2 corrector and said second C corrector is a C1 corrector.

In another embodiment, the kit may further include a collection of components and/or agents present in single or separate compositions for analyzing mutant CFTR. More particular such collection is used to analyze the sequence of CFTR protein from the subject for the presence of mutations between amino acid residues 1164-1480 of the wild type CFTR. More particular such collection is used to analyze for the presence of a premature termination codon (PTC) or a nonsense mutation in said region.

The kit may include instructions for practicing the methods and using the combinations of the invention, and, optionally, for performing a diagnostic assay, such as an informational package insert describing the use and attendant benefits of the drugs in treating CF. These instructions may be present in the kits in a variety of forms, one or more of which may be present in or on the kit.

### Method of Enhancing the Activity of Mutant CFTR

The present invention further provides a method of enhancing the activity of mutant CFTR having a mutation located between the amino acid residues 1164-1480 of SEQ ID NO: 1 in a cell, comprising the step of contacting said cell with a combination comprising:
i. a modulator of the function (P potentiator) of cystic fibrosis transmembrane conductance regulator (CFTR) protein,
ii. a modulator of the cellular processing and/or localization molecule (C corrector), wherein said C corrector is not a read-through corrector,
   wherein said combination does not comprise a read-through agent, and wherein said combination produces an additional transepithelial conductance (ΔGt) of at least 1 mS/cm2 as measured using transepithelial clap circuit assay in the W1282X Fisher rat thyroid (FRT) cells.

In alternative embodiment the present invention provides a method of enhancing the activity of mutant CFTR having a mutation located between the amino acid residues 1164-1480 of SEQ ID NO: 1 in a cell, comprising the step of contacting said cell with a combination comprising:
i. a modulator of the function (P potentiator) of cystic fibrosis transmembrane conductance regulator (CFTR) protein,
ii. a modulator of the cellular processing and/or localization molecule (C corrector), wherein said C corrector is not a read-through corrector, wherein said corrector is not acting through the membrane spanning domain 1 (MSD1) of CFTR,
   wherein said combination does not comprise a read-through agent.

In alternative embodiment the present invention provides a method of enhancing the activity of mutant CFTR having a mutation located between the amino acid residues 1164-1480 of SEQ ID NO: 1 in a cell, comprising the step of contacting said cell with a combination comprising:
i. a modulator of the function (P potentiator) of cystic fibrosis transmembrane conductance regulator (CFTR) protein,
ii. a modulator of the cellular processing and/or localization molecule (C corrector), wherein said C corrector is not a read-through corrector, wherein said corrector is acting through the membrane spanning domain 1 (MSD1) of CFTR,
   wherein said combination does not comprise a read-through agent.

In a particular embodiment said methods are performed ex vivo. In yet another embodiment said method is performed in vivo.

In a particular embodiment said combination further comprises a second modulator of the cellular processing and/or localization (second C corrector), wherein said second C corrector is not a read-through corrector.

In another particular embodiment said CFTR protein comprises a premature termination codon (PTC) or a nonsense mutation, and wherein said mutation is located between the amino acid residues 1164-1480 of SEQ ID NO: 1

In a specific embodiment the premature termination codon (PTC) or a nonsense mutation is UGA codon (or opal codon).

In more specific embodiment said mutation in CFTR is W1282X mutation.

In a particular embodiment C corrector is a C1 corrector as described herein. In yet another embodiment said C corrector is a C2 corrector as described herein. In another embodiment said C corrector is a C2 corrector as described herein and said second C corrector is a C1 corrector as described herein.

The invention is further illustrated in the following examples. These examples should not be considered limiting and are provided to assist the skilled person in performing the invention.

### Examples

### Example 1. Effect of the Potentiation and Corrector Combinations on Class I Mutations (“Acute” Protocol)

### Plasmid Construction

The CFTR W1282X gene was inserted into Fisher Rat thyroid cells using the Flp-inTM system (Invitrogen). Briefly, the plasmid pFRT/Lac ZEO is stably transfected into the FRT cell line to generate a Zeocin resistant Flp-In host cell. The pcDNA5/FRT plasmid containing CFTR W1282X is co-transfected with pOG44 into the host Flp-In cell line. The Flp-In recombinase expressed by pOG44 catalyzes a homologous recombination event between the FRT sites of the host cells and the pCDNA5/FRT expression vector. Integration of the expression construct allows expression of CFTR W1282X and confers hygromycin resistance and zeocin sensitivity to the cells.

### Mammalian Cell Culture and Transfection

Fischer Rat Thyroid (FRT) cells were cultured in Ham's F-12 medium (Sigma) supplemented with 5% FBS and 2.68 g/L sodium bicarbonate (Sigma).

### Transepithelial Conductance (Gt) Measurements of FRT Cell Monolayers

Cells were grown to confluence on costar 24 well 0.4 M permeable supports and treated with Correctors (C1 (0.5 uM) and/or C2 (3 uM)) for 48 hrs. Prior to drug treatment, the transepithelial resistance of the cells was measured using epithelial voltmeter (EVOM², EMD Millipore), which was in the range of 8-10 kS2 cm².

Transepithelial conductance of the FRT cells was measured using conductance machine (PrecisePlace 2300 Robot, Precision Automation Inc.) (“Acute” protocol) Briefly the cells were treated during 24 hours with C1 and/or C2 and/or G418. The day after, cells were placed in bicarbonate free Ham's F-12 coon's media (Sigma) with preincubation at 37° C. for 30 mins. The baseline conductance measurements of the epithelial monolayer were recorded for 12 mins followed by the stimulation of CFTR activity by addition of 100 nM or 10 μM forskolin and then with 10 uM potentiators to the apical and basolateral surface of the cells. Finally CFTR_{Inh-} 172 (10 μM) was added to the apical surface to block the CFTR dependent conductance.

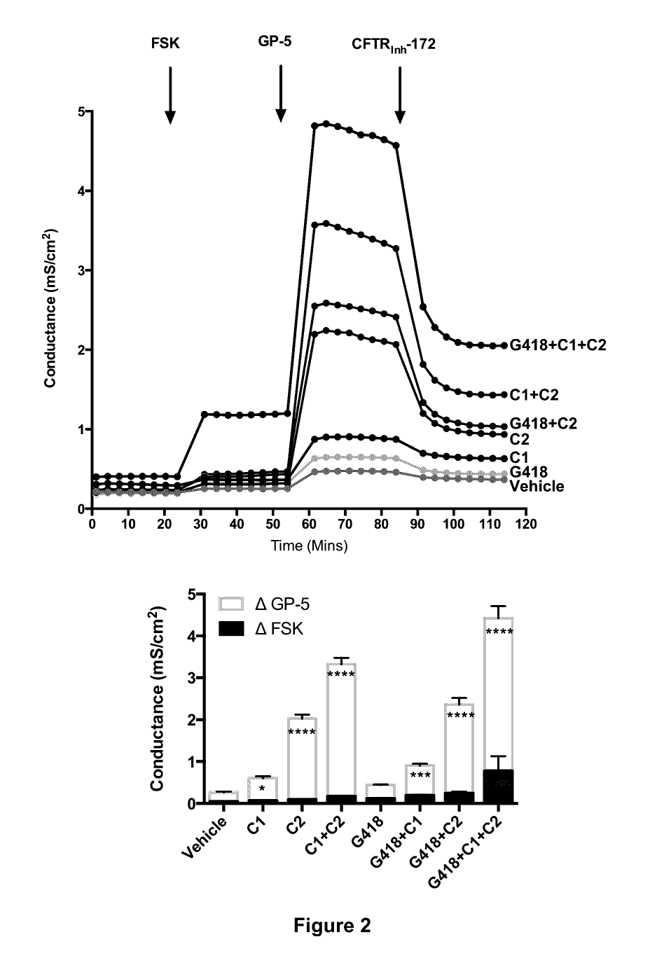

The results are presented in the FIG. 2.

### Example 2. TECC Assay in Primary Bronchial Epithelial Cells

The TECC (Tranepithelial Clamp Circuit, EP-design) assay measures the functionality of the cystic fibrosis Transmembrane Conductance regulator (CFTR) by measuring the short circuit current (I_{sc}) generated over the basolateral and apical membrane of lung epithelial cells. In TECC the transepithelial potential PD and transepithelial resistance (Rₜ) are measured in an open circuit and transformed to I_{sc} using Ohm's law. 24 wells can be measured simultaneously allowing a higher throughput compared to Ussing chambers.

For this purpose, bronchial epithelial cells isolated from CF patients homozygous for the CFTR ΔF508 mutation (hAEC-CF, Epithelix, Geneva, Switzerland; McGill University, Montreal, Qc; Asterand, Detroit, Mich.; University of North Carolina, Chapel Hill, N.C.) are plated on type IV collagen-coated Transwell supports (Costar). Human airway epithelia are generated by provision of an air-liquid interface for 21 days to form well-differentiated polarized cultures that resemble in vivo pseudo-stratified ciliated epithelium (Fulcher et al., 2005). The differentiated cells are treated with test corrector compounds (“acute”) or test corrector compounds and potentiator GLPG1837 (“Chronic”) for 24 hours basolaterally to allow sufficient expression of properly folded CFTR protein on the membrane.

For electrophysiological recording of the “acute” experiments, the human airway epithelia are mounted in the TECC heating plate and kept at 37° C. The epithelia are bathed in a NaCl-Ringer solution (120 mM NaCl, 25 mM NaHCO₃, 1.2 mM CaCl₂, 1.2 mM MgCl₂, 0.8 mM KH₂PO₄, 0.8 mM K₂HPO₄, pH 7.4, 5 mM glucose) on both the basolateral and apical sides. Test compounds are re-added to the recording solution prior to measurement. Apical amiloride is used to inhibit the endogenous ENaC currents while forkolin is applied on both apical and basolateral side to stimulate CFTR. CFTR activity is measured by addition of forskolin followed by addition of a potentiator, GLPG1837, on both sides. Measurements are done during a 20 minute timeframe with recordings every 2 minutes. The increase in I_{sc} is used as a measure for the increased CFTR activity, EC₅₀ values can be generated by measuring impact of different concentrations of compound on I_{sc} on primary cells, for this purpose each transwell is treated with a different compound concentration for 24 hours. Inh-172, an inhibitor specific for CFTR, is used to test the specificity of the tested compounds.

For electrophysiological recording of the “chronic” experiments, the human airway epithelia are mounted in the TECC heating plate for electrophysiological measurement and kept at 37° C. The epithelia are bathed in a NaCl-Ringer solution (120 mM NaCl, 25 mM NaHCO₃, 1.2 mM CaCl₂, 1.2 mM MgCl₂, 0.8 mM KH₂PO₄, 0.8 mM K₂HPO₄, pH 7.4, 5 mM glucose) on both the basolateral and apical sides. Test compounds (corrector and potentiator GLPG1837) are re-added to the recording solution prior to measurement. Apical amiloride is used to inhibit the endogenous ENaC currents while forkolin is applied on both apical and basolateral side to stimulate CFTR. Measurements are done during a 20 minute timeframe with recordings every 2 minutes. The increase in I_{sc} is used as a measure for the increased CFTR activity, EC₅₀ values can be generated by measuring impact of different concentrations of compound on I_{sc} on primary cells, for this purpose each transwell is treated with a different compound concentration. Inh-172, an inhibitor specific for CFTR, is used to test the specificity of the tested compounds.

Information on protein binding of compounds can be retrieved from incubation of compounds in presence of 40% human serum. For this purpose the differentiated cells are treated basolaterally with test compounds in medium containing 40% human serum (Sigma; H4522) for 24 hours. For electrophysiological recording, the human airway epithelia are mounted in the TECC heating plate and kept at 37° C. The epithelia are bathed in a NaCl-Ringer solution (120 mM NaCl, 25 mM NaHCO₃, 1.2 mM CaCl₂, 1.2 mM MgCl₂, 0.8 mM KH₂PO₄, 0.8 mM K₂HPO₄, pH 7.4, 5 mM glucose) on both the basolateral and apical sides. Test compounds (corrector and potentiator GLPG1837) are re-added to the recording solution prior to measurement. Apical amiloride is used to inhibit the endogenous ENaC currents while forkolin is applied on both apical and basolateral side to stimulate CFTR. Measurements are done during a 20 minute timeframe with recordings every 2 minutes. The increase in I_{sc} is used as a measure for the increased CFTR activity, EC₅₀ values can be generated by measuring impact of different concentrations of compound on I_{sc} on primary cells, for this purpose each transwell is treated with a different compound concentration. Inh-172, an inhibitor specific for CFTR, is used to test the specificity of the tested compounds.

### Example 3. Measuring CFTR Cell Surface Levels Using HRP-Tagged ΔF508-CFTR Expressing CFBE Cells

The HRP-tagged ΔF508-CFTR cell assay measures the expression of CFTR-ΔF508 at the plasma membrane. CFTR-ΔF508 has a folding defect leading to absence of protein at the plasma membrane. This assay is used to evaluate the capacity of compounds to increase the expression of CFTR-ΔF508 at the plasma membrane. The CFTR-ΔF508 is tagged with HRP (Horse radish Peroxidase enzyme) within the ECL4 (Extracellular loop 4) of CFTR. When HRP-tagged ΔF508-CFTR is present at the plasma membrane, the HRP enzyme activity can be measured. The amount of CFTR-ΔF508 that can be rescued to the plasma membrane is correlated with the amount of functional enzyme that can be measured.

There are several ways to measure the capacity of compounds to rescue CFTR-ΔF508 to the plasma membrane; either compounds are evaluated on their own and the impact on plasma membrane levels is measured or compounds are evaluated in combination with a co-corrector i.e. a compound that rescues CFTR-ΔF508 to the plasma membrane but rescue can be enhanced by addition of compounds due to complementary mode of action.

### Activity of Corrector Compounds in Combination with an Additional Corrector

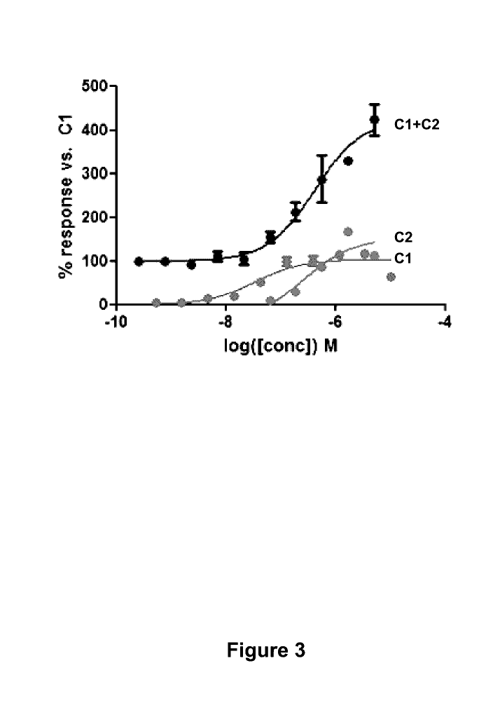

For this purpose Doxycycline-inducible ΔF508-CFTR-HRP expressing CFBE41o- cells (obtained from Gergely Lukacs, McGill University) were maintained in MEM (Gibco; 31095) supplemented with 10% fetal bovine serum (Hyclone; SV30160.03) under puromycin (3 μg/ml) and G418 selection (0.2 mg/ml). For compound testing, cells were seeded at 4000 cells/well in white 384 well plates (Greiner; 781080) in 50 μL medium containing 0.5 μg/ml doxycycline and incubated for 68 hours at 37° C., 5% CO₂. On day four, 10 μl test compounds diluted in PBS were added to the plates at a final DMSO concentration of 0.1%. In order to measure compound synergy with a co-corrector, 3 μM co-corrector was added along with test compounds. All compound plates contained negative controls (DMSO) and positive controls (3 μM co-corrector). Cell plates were incubated at 33° C., 5% CO₂ for 20 hours. On day five, the cells were washed five times with phosphate-buffered saline, and HRP activity was assayed by the addition of 50 μL/well of HRP substrate (SuperSignal West Pico Chemiluminescent Substrate, Thermo Scientific; 34080). After incubation for 15 minutes in the dark, chemiluminescence was measured using a plate reader (EnVision, Perkin Elmer). Raw data were normalized to percentage activity values using the equation: 100×(Sample−Negative control)/(Positive control−Negative Control). The results for the combination of C1 and C2 are presented in FIG. 3, where the concentration of C1 was kept constant.

### Activity of Corrector as their Intrinsic Corrector Capacity

For this purpose Doxycycline-inducible ΔF508-CFTR-HRP expressing CFBE41o- cells (obtained from Gergely Lukacs, McGill University) were maintained in MEM (Gibco; 31095) supplemented with 10% fetal bovine serum (Hyclone; SV30160.03) under puromycin (3 μg/ml) and G418 selection (0.2 mg/ml). For compound testing, cells were seeded at 4000 cells/well in white 384 well plates (Greiner; 781080) in 50 μL medium containing 0.5 μg/ml doxycycline and incubated for 68 hours at 37° C., 5% CO₂. On day four, 10 μl test compounds diluted in PBS were added to the plates at a final DMSO concentration of 0.1%. All compound plates contained negative controls (DMSO) and positive controls (3 μM co-corrector). Cell plates were incubated at 33° C., 5% CO₂ for 20 hours. On day five, the cells were washed five times with phosphate-buffered saline, and HRP activity was assayed by the addition of 50 μL/well of HRP substrate (SuperSignal West Pico Chemiluminescent Substrate, Thermo Scientific; 34080). After incubation for 15 minutes in the dark, chemiluminescence was measured using a plate reader (EnVision, Perkin Elmer). Raw data were normalized to percentage activity values using the equation: 100×(Sample−Negative control)/(Positive control−Negative Control). The results of the effects of both C1 and C2 correctors separately are presented in FIG. 3 where 100% response corresponds to the maximum level obtained with C1 alone.

### Example 4. YFP-Halide Influx Assay for the CFTR-ΔF508 Mutation

The YFP halide influx assay measures the functionality of the Cystic Fibrosis Transmembrane Conductance regulator (CFTR) channels in the cystic fibrosis bronchial epithelium cell line CFBE41o-. The fluorescence of the yellow fluorescent protein (YFP) variant YFP H148Q, 1152L or variant YFP H148Q, 1152L & F47L is substantially quenched by iodine, a halide that is efficiently transported by CFTR. The assay is thus used to evaluate the effect of corrector compounds on CFTR channel function by measuring the extent of YFP signal quenching. (Galietta et al., 2001; Nagai et al., 2002)

For this purpose, CFBE41o- cells are seeded in 96 well plates (6000 CFBE cells/well). One day after seeding, the CFBE cells are transduced with adenoviral vectors that direct the expression of the CFTR ΔF508 mutant and of the YFP reporter. Cells are treated with test compounds for 24 h at 37° C. to allow trafficking of corrected CFTR to the membrane.

The next day the CFTR channels are activated by treatment with the cAMP inducer forskolin (10.67 μM) and potentiator GLPG1837 (0.5 μM) in 1×D-PBS (from Gibco, Cat n#14090-091) for 20 minutes prior to addition of an I⁻ solution (137 mM NaI, 2.7 mM KI, 1.76 mM KH₂PO₄, 10.1 mM Na₂HPO₄, 5 mM glucose). The I⁻ induced quenching of fluorescence is recorded immediately after injection of I⁻ for 7 seconds. The capacity of a compound to increase number of channels, and therefore overall halide influx is directly correlated with the decrease in fluorescence, and is expressed as (1−(fluorescence after 7 seconds (F)/fluorescence before injection (F0))) and an EC₅₀ can be derived from a (1−F/F0) vs compound concentration plot.

### Example 5. Measuring Binding Using Molecular Sensing Technology

TruBind™ Back-Scattering Interferometry (BSI) (Molecular Sensing GmbH) can be used to determine binding constants of different compounds to CFTR-expressing cells.

HEK293 wild type CFTR and HEK293 control membrane fractions are used as 100 μg (total protein amount) aliquots in 50 mM Tris-HCl pH 7.5, 1 mM EDTA, 10% Glycerol+PIC and stored at −80° C.

The buffer used for the assays is 50 mM Tris-HCl pH 7.5, 1 mM EDTA with 1.2% DMSO. The refractive index of the assay buffer and the compound are matched and then a 2x serial dilution is done in polypropylene dilution reservoirs.

A thawed aliquot of HEK293.CFTRwt as well as a thawed aliquot of HEK293 control membrane fractions is diluted to 10 mL in 50 mM Tris-HCl pH 7.5, 1 mM EDTA with 1.2% DMSO. The refractive index of the assay buffer and the two membrane fractions are matched by adding water to the membrane fractions.

Compound and target are mixed 1:1 in 96-well PCR microtiter plates to a final volume of 150 μL and heat sealed with foil. The assays are allowed to incubate at room temperature for 4 hours before being run on the BSI instrument. Wells are pierced individually prior to sample injection and measurement of BSI signal (each well is analyzed in quadruplicates).

The chip fluidic channels are coated with hybrid bilayer membranes (HBM) (Molecular Sensing GmbH). Before each assay, a fresh HBM layer is applied. A basement layer is created suitable for capture of the HBM reagent. HBM reagents were flown through each channel for 15 min followed by an injection of the assay buffer to remove loosely adhered lipid layers.

The BSI system is used in a Dual Channel mode injecting in parallel. This allows the measurement of the binding affinity between compound and target, the wild-type CFTR membrane fractions (assay), at the same time as unspecific binding between compound and control membrane fractions (reference). For each assay the reference data is subtracted from the assay data. The resulting difference signal is compared to two different controls, which are the serial dilution of the compound alone and the target alone (wild-type CFTR membrane fractions in one channel and control membrane fractions in the other channel).

The final data for the difference curve is exported to Graphpad Prism® and analyzed using a one-site binding equation to determine a K_{d} for the assay. Success is defined as having a binding signal with a correlation coefficient of at least 0.7.

The K_{d} values have been determined for correctors of C1 and C2 types. The results for exemplary C2 correctors and an exemplary C1 corrector are presented in table 1. It can be seen that C1 corrector has binding affinity similar to the known correctors acting on CFTR. C2 correctors do not demonstrate significant binding affinity to the membrane fraction of CFTR.

TABLE 1
K_{d} values for exemplary C1 and C2 compounds.

| Compound | K_{d} (nM) | R² |
|---|---|---|
| C1 | 94.7 ± 26.5 | 0.72 |
| C2 | 483.6 ± 121.2 | 0.79 |
| C2 | 8512 ± 2309 | 0.81 |
| C2 | 685.2 ± 121.6 | 0.87 |

### Example 6. Effect of the Potentiation and Corrector Combinations on Class I Mutations (“Chronic” Protocol)

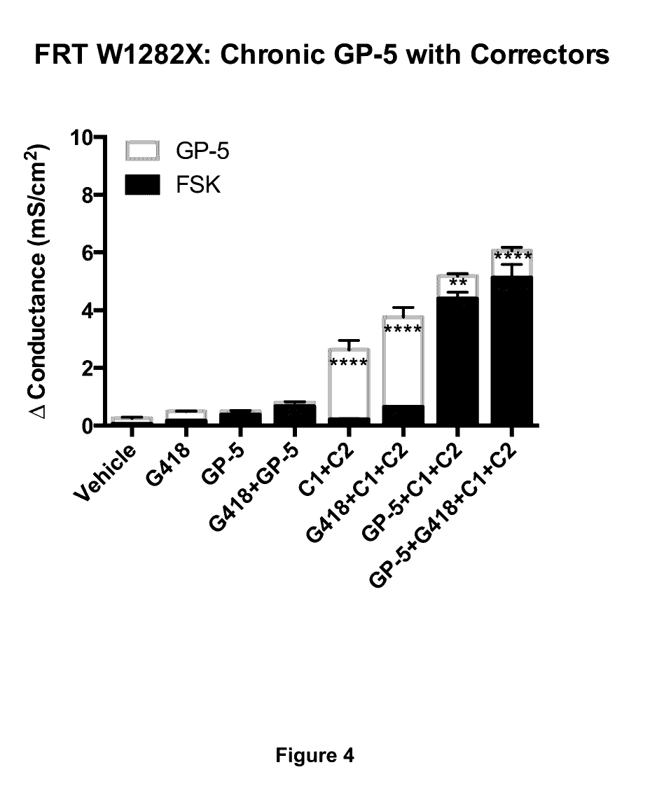

An alternative to this protocol can be done as following (“Chronic” protocol), Transepithelial conductance of the FRT cells was measured using conductance machine (PrecisePlace 2300 Robot, Precision Automation Inc.) Briefly the cells were treated during 24 hours with C1 and/or C2 and/or G418 and GP-5 potentiator. The day after, cells were placed in bicarbonate free Ham's F-12 coon's media (Sigma) with preincubation at 37° C. for 30 mins. The baseline conductance measurements of the epithelial monolayer were recorded for 12 mins followed by the stimulation of CFTR activity by addition of 100 nM or 10 μM forskolin to the apical and basolateral surface of the cells. Finally CFTR_{Inh-} 172 (10 μM) was added to the apical surface to block the CFTR dependent conductance. The results comparing the “acute” and “chronic” effects of potentiator are presented in FIG. 4.

### Example 7. TECC Assay in ΔF508/W1282X Primary Bronchial Epithelial Cells

The TECC (Tranepithelial Clamp Circuit, EP-design) assay measures the functionality of the cystic fibrosis Transmembrane Conductance regulator (CFTR) by measuring the short circuit current (I_{sc}) generated over the basolateral and apical membrane of lung epithelial cells. In TECC the transepithelial potential PD and transepithelial resistance (Rₜ) are measured in an open circuit and transformed to I_{sc} using Ohm's law. 24 wells can be measured simultaneously allowing a higher throughput compared to Ussing chambers.

For this purpose, bronchial epithelial cells isolated from CF patients harboring F508del mutation on one allele and W1282X on the other allele are plated on type IV collagen-coated Transwell supports (Costar). Human airway epithelia are generated by provision of an air-liquid interface for 21 days to form well-differentiated polarized cultures that resemble in vivo pseudo-stratified ciliated epithelium (Fulcher et al., 2005). The differentiated cells are treated with test corrector compounds C1/C2 and/or G418 for 24 hours basolaterally to allow sufficient expression of properly folded CFTR protein on the membrane.

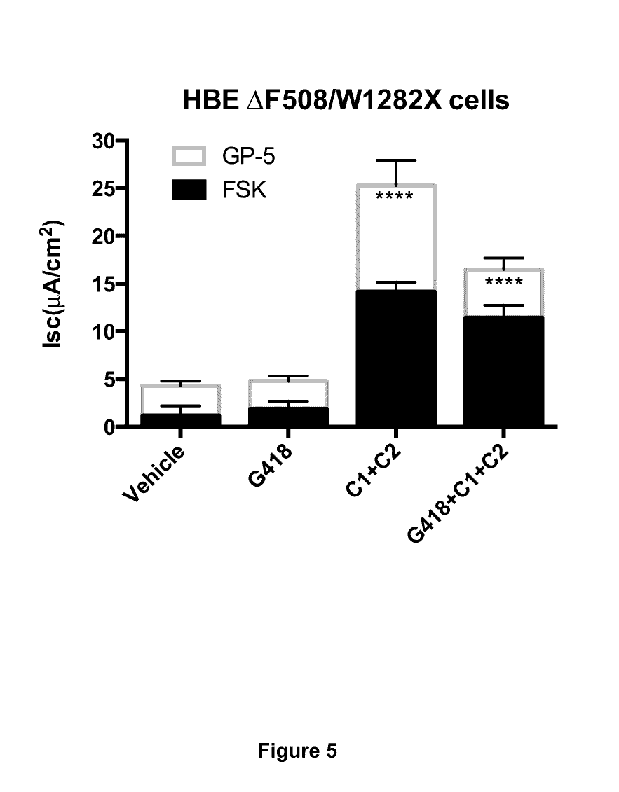

For electrophysiological recording, the human airway epithelia are mounted in the TECC heating plate and kept at 37° C. The epithelia are bathed in a NaCl-Ringer solution (120 mM NaCl, 25 mM NaHCO₃, 1.2 mM CaCl₂, 1.2 mM MgCl₂, 0.8 mM KH₂PO₄, 0.8 mM K₂HPO₄, pH 7.4, 5 mM glucose) on both the basolateral and apical sides. Test compounds are re-added to the recording solution prior to measurement. Apical amiloride is used to inhibit the endogenous ENaC currents while forkolin is applied on both apical and basolateral side to stimulate CFTR. CFTR activity is measured by addition of forskolin followed by addition of a potentiator, GP-5, on both sides. Measurements are done during a 20 minute timeframe with recordings every 2 minutes. The increase in I_{sc} is used as a measure for the increased CFTR activity. Inh-172, an inhibitor specific for CFTR, is used to test the specificity of the tested compounds. FIG. 5 shows rescue of W1282X/F508del CFTR using corrector molecules and/or readthrough agents combined with GP-5 potentiator.

### Example 8. CFTR Western Blot Analysis

The protocol used for the western blot was essentially the one disclosed in Xue et. al., 2014. In short, the FRT cells were treated during 24 hours with C1 and/or C2 and/or G418 and GP-5 potentiator. The cells were harvested on day 1. For that the cells were rinsed with cold PBS and collected with cold PBS. The collected cells were subsequently centrifuged at 4° C. for 2 min at 12,000 rpm. If necessary the resulting pellet can be stored at −80° C. on day 2 the pellerts were lysed on ice for 45 min using Native Lysis Buffer (50 mM Tris-HCl pH 8.5, 150 mM NaCl and 1% NP-40) containing 10% ethylenediaminetetraacetic acid (EDTA) and 10% protease inhibitor (PI, Thermoscientific, Waltham, Mass.) vortexing briefly every 5 min. The lysate was centrifuged at 12,000 rpm at 4° C. for 10 min and was transferred into tubes.

The protein amount in the tubes was quantified using a BCA assay kit. FRT cell lysates were normalized for protein concentration and separated by gel electrophoresis.

Equal amounts of protein were electrophoresed on SDS-PAGE gels (Invitrogen, Carlsbad, Calif.) then transferred to nitrocellulose membranes (BioRad Laboratories, Hercules, Calif.). Blots were blocked in 1x PBS containing 5% (w/v) milk powder and 0.1% Tween-20, then incubated with 1:5000 diluted anti-CFTR antibody (1:1 mixture of 570 and 596 monoclonal anti-CFTR antibodies) (CFFT therapeutics Inc) for 2 hours at room temperature, washed, followed by secondary goat-anti-mouse antibody (Dako, Carpinteria, Calif.) conjugated with horseradish peroxidase (1:10,000) for 1 h at room temperature. Chemiluminescence was induced with high-sensitivity West Femto High Sensitivity Substrate (Thermo). The membranes were exposed using CemiDoc XRS HQ (Bio-Rad, Hercules, Calif., USA) for different periods (up to 2 min) and calibrated in the linear range for a standard set of diluted samples

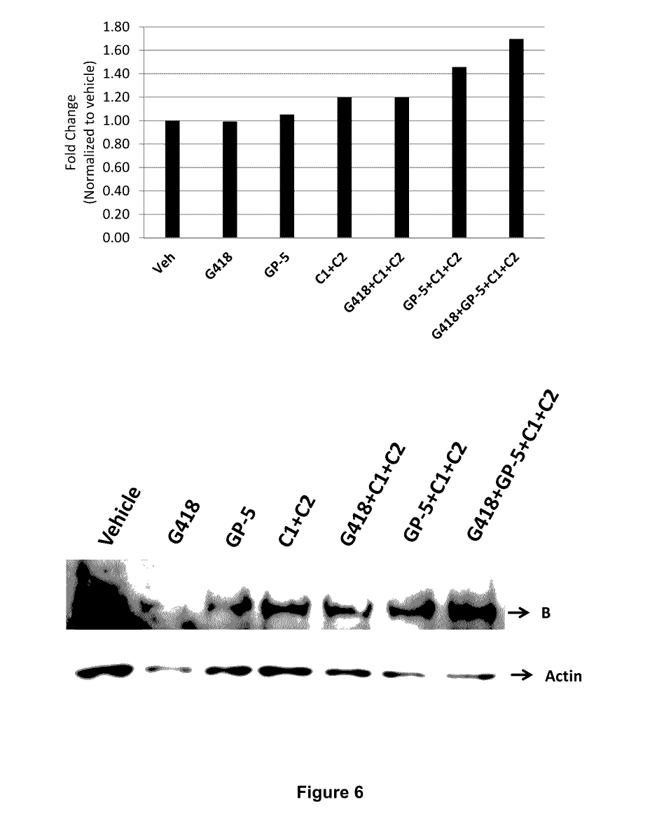

The CFTR protein levels in the presence of different potentiator/corrector(s) combinations using this protocol are presented in FIG. 6. It can be seen that the combination of C1 and C2 alone produces the same or higher level of CFTR produced in the cells as in the presence of a read.

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

### REFERENCES

Audrezet et al (2004) Hum Mutat. April; 23(4):343-57
Becq F, Mall M A, Sheppard D N, Conese M, Zegarra-Moran O. (2011) J Cyst Fibros. 10:S129-45
Flume P A, Liou T G, Borowitz D S, Li H, Yen K, Ordonez C L, Geller D E (2012) Chest Mar 1
Gusev et al. (2001) Am J Pathol 159:63
Gregory, R. J. et al. (1990) Nature 347:382-386;
Hermann T (2007) Cell Mol Life Sci. 64(14):1841-52
Howard M, Frizzell R A, Bedwell D M (1996) Nat Med. 2(4):467-9
Kerem B, Chiba-Falek O, Kerem E (1997) Genet Test. 1997; 1(1):35-9.
Kerem, Hirawat, Armoni, Yaakov, Shoseyov, Cohen, et al. (2008) Lancet. 372(9640):719-727
Lieder (2000) Advance for Laboratory Managers, 70
MacDonald K D, McKenzie K R, Zeitlin P L (2007) Paediatr Drugs 9:1-10.
Marras et al. (1999) Genet Anal 14:151
Rich, D. P. et al. (1990) Nature 347:358-362
Riordan J R. (2005) Annu Rev Physiol. 67:701-18
Rogan M P, Stoltz D A, Hornick D B. (2011) Chest 139: 1480-1490
Rowe and Verkman (2013) Cold Spring Harbor Perspectives in Medicine 3(7).
Rowe et al, 2007
Van Goor F, Hadida S, Grootenhuis P D, et al. (2009) Proc Natl Acad Sci USA 106: 18825-18830
Wilschanski, Yahav, Yaacov, Blau, Bentur, Rivlin, et al. (2003) N Engl J Med. 349(15):1433-1441;
Wilschanski (2012) Front. Pharmacol. 3:117. doi: 10.3389/fphar.2012.00117 www.clinicaltrials.gov: Study of VX-809 Alone and in Combination With VX-770 in Cystic Fibrosis (CF) Patients Homozygous or Heterozygous for the F508del-CFTR Mutation, study code NCT0122521 1
Xue et al (2014) Am J Respir Cell Mol Biol. 50(4): 805-816.

We claim:

1. A method of treatment of cystic fibrosis in a subject comprising the steps of:
a) analyzing the sequence of cystic fibrosis transmembrane conductance regulator (CFTR) protein from the subject for the presence of a premature termination codon (PTC) or a nonsense mutation,
b) identifying a subject having a mutation located between the amino acid residues 1164-1480 of SEQ ID NO: 1, and
c) administering a combination comprising:
i. a modulator of the function (P potentiator) of cystic fibrosis transmembrane conductance regulator (CFTR) protein,
ii. a modulator of the cellular processing and/or localization (C corrector), wherein said C corrector is not a read-through corrector, wherein said corrector is not acting through the membrane spanning domain 1 (MSD1) of CFTR,
and wherein said combination does not comprise a read-through agent

2. The method of treatment of cystic fibrosis according to claim 1, wherein the cystic fibrosis results from a Class I mutation in CFTR protein, wherein said CFTR protein comprises a premature termination codon (PTC) or a nonsense mutation, and wherein said mutation is located between the amino acid residues 1164-1480 of SEQ ID NO: 1.

3. The method of claim 1, wherein the short circuit (I_{sc}) current as measured by the TECC assay on F508del homozygous patient derived cells using said combination yields at least 15% of the I_{sc} obtained with the CFTR protein according to SEQ ID NO: 1 as measured by the TECC assay.

4. The method of treatment of cystic fibrosis according to claim 1, wherein said corrector binds to CFTR protein.

5. The method of treatment of cystic fibrosis according to claim 1, wherein said C corrector does not bind to the MSD1 domain of the CFTR protein.

6. The method of treatment of cystic fibrosis according to claim 1, where said combination additionally comprises a second modulator of the cellular processing and/or localization (second C corrector), wherein said second C corrector is not a read-through corrector.

7. The method of treatment of cystic fibrosis according to claim 6, wherein said second C corrector binds to the CFTR protein.

8. The method of treatment of cystic fibrosis according to claim 6, wherein said first corrector and the second C corrector bind to different portions of the CFTR protein.

9. The method of treatment of cystic fibrosis according to claim 6, wherein said second C corrector acts through the MSD1 domain of the CFTR protein.

10. The method of treatment of cystic fibrosis according to claim 6, wherein said second C corrector binds to the MSD1 domain of the CFTR protein.

11. The method of treatment of cystic fibrosis according to claim 6, wherein said correctors act via different mechanisms.

12. The method of treatment of cystic fibrosis according to claim 8, wherein said binding is measured using transepithelial clap circuit assay (TECC assay) and Molecular Sensing technology.

13. The method of treatment of cystic fibrosis according to claim 1, wherein said combination produces an additional transepithelial conductance (ΔGt) of at least 1 mS/cm2 as measured using transepithelial clap circuit assay in the W1282X Fisher rat thyroid (FRT) cells.

14. The method of treatment of cystic fibrosis according to claim 6, wherein said combination produces an additional transepithelial conductance (ΔGt) of at least 3.5 mS/cm2 as measured using transepithelial clap circuit assay (TECC assay) in the W1282X Fisher rat thyroid (FRT) cells.

15. The method of treatment of cystic fibrosis according to claim 1, wherein the short circuit (I_{sc}) current as measured by the TECC assay on F508del homozygous patient derived cells using said combination yields at least 30% of the I_{sc} obtained with the CFTR protein according to SEQ ID NO: 1 as measured by the TECC assay.

16. The method of treatment of cystic fibrosis according to claim 6, wherein the short circuit (I_{sc}) current as measured by the trans epithelial clamp circuit assay (TECC assay) using the combination is at least equal to 85% of the sum of the individual I_{sc} of the each correctors in the same cells.

17. The method according to claim 1, wherein the premature termination codon (PTC) or a nonsense mutation is UGA codon (or opal codon).

18. The method according to claim 1, wherein said mutation is W1282X mutation.

19. The method of treatment of cystic fibrosis according to claim 1, wherein said C corrector is C2 corrector.

20. The method of treatment of cystic fibrosis according to claim 6, wherein said second C corrector is C1 corrector.

21. The method of treatment of cystic fibrosis according to claim 1, wherein said P potentiator is a compound according to formula (I) or formula (II), or a pharmaceutically acceptable salt thereof.

22. The method of treatment of cystic fibrosis according to claim 1, wherein said C corrector is a compound according to formula (IV), formula(V), or a pharmaceutically acceptable salt thereof.

23. The method of treatment of cystic fibrosis according to claim 6, wherein said second C corrector is a compound according to formula (III), or a pharmaceutically acceptable salt thereof.

24. The method according to claim 1 wherein the P potentiator molecule is selected from

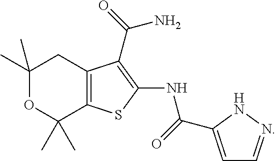 [SMILES: CC1(C)Cc2c(sc(NC(=O)c3ccn[nH]3)c2C(N)=O)C(C)(C)O1]

25. The method according to claim 1, wherein the C corrector molecule is

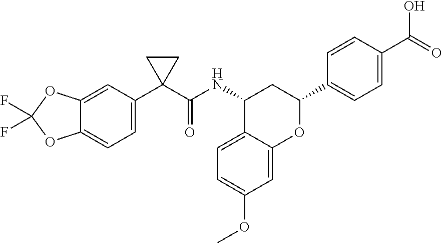 [SMILES: COc1ccc2c(c1)O[C@@H](c1ccc(C(=O)O)cc1)C[C@H]2NC(=O)C1(c2ccc3c(c2)OC(F)(F)O3)CC1]

26. The method according to claim 6, wherein said C corrector is 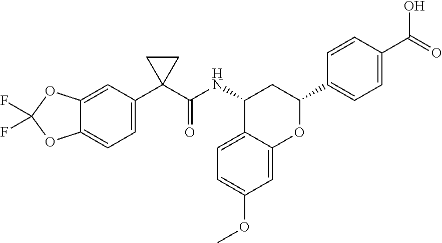 [SMILES: COc1ccc2c(c1)O[C@@H](c1ccc(C(=O)O)cc1)C[C@H]2NC(=O)C1(c2ccc3c(c2)OC(F)(F)O3)CC1]
and said second C corrector is selected from the compounds according to formula (IV) and formula (V), or a pharmaceutically acceptable salt thereof.

27. A method of enhancing the activity of mutant CFTR having a mutation located between the amino acid residues 1164-1480 of SEQ ID NO: 1 in a cell, comprising the step of contacting said cell with a combination comprising:
i. a modulator of the function (P potentiator) of cystic fibrosis transmembrane conductance regulator (CFTR) protein,
ii. a modulator of the cellular processing and/or localization molecule (C corrector), wherein said C corrector is not a read-through corrector, wherein said corrector is not acting through the membrane spanning domain 1 (MSD1) of CFTR,
wherein said combination does not comprise a read-through agent.

28. The method according to claim 27, wherein said combination further comprises a second modulator of the cellular processing and/or localization (a second C corrector), wherein said second C corrector is not a read-through corrector.

29. The method according to claim 27, wherein said CFTR protein comprises a premature termination codon (PTC) or a nonsense mutation, and wherein said mutation is located between the amino acid residues 1164-1480 of SEQ ID NO: 1.

30. The method according to claim 27, wherein said cell is ex vivo.

31. The method according to claim 27, wherein said cell is in vivo.

32. The method according to claim 27, wherein the premature termination codon (PTC) or a nonsense mutation is UGA codon (or opal codon).

33. The method according to claim 27, wherein said mutation is W1282X mutation.

34. The method according to claim 28, wherein said C corrector and the second C corrector bind to different portions of the CFTR protein.

35. The method according to claim 28, wherein the said correctors act via different mechanisms.

36. The method according to claim 28, wherein said one of the correctors binds to MSD1 domain of the CFTR protein, and wherein another corrector does not bind to MSD1 domain.

37. The method according to claim 27, wherein the premature termination codon (PTC) or a nonsense mutation is UGA codon (or opal codon).

38. A kit comprising:
i. a pharmaceutical composition comprising a P potentiator;
ii. a pharmaceutical composition comprising a C corrector, wherein said C corrector is not a read-through corrector, wherein said corrector is not acting through the membrane spanning domain 1 (MSD1) of CFTR;
iii. instructions for using said kit for treating cystic fibrosis in a subject having a mutation located between the amino acid residues 1164-1480 of SEQ ID NO: 1,
wherein said kit does not comprise a read-through agent.

39. The kit according to claim 38, wherein said kit further comprises a second modulator of the cellular processing and/or localization (second C corrector), wherein said second C corrector is not a read-through corrector.

40. The kit according to claim 39, wherein said correctors bind to different portions of the CFTR protein.

41. The kit according to claim 39, wherein the said correctors act via different mechanisms.

42. The kit according to claim 39, wherein said one of the correctors bind to MSD1 domain of the CFTR protein, and wherein another corrector does not bind to MSD1 domain.
